(19) United States
(12) Patent Application Publication
(10) Pub. No.: US 2024/0068004 A1
(43) Pub. Date: Feb. 29, 2024

(54) CELL-FREE METHODS OF PRODUCING ANTIBODIES TO INTRACELLULAR TARGETS

(71) Applicant: MBrace Therapeutics, Inc., San Diego, CA (US)

(72) Inventors: Yongjian Wu, San Diego, CA (US); Fernanda I. Staquicini, Jersey City, NJ (US); Isan Chen, San Diego, CA (US)

(73) Assignee: MBrace Therapeutics, Inc., San Diego, CA (US)

(21) Appl. No.: 18/311,235

(22) Filed: May 2, 2023

Related U.S. Application Data
(63) Continuation of application No. 63/398,143, filed on Aug. 15, 2022.

(51) Int. Cl. C12P 21/00; C07K 16/18; C07K 16/40

(52) U.S. Cl. CPC C12P 21/005; C07K 16/18; C07K 16/40

(57) ABSTRACT

Provided herein are methods and cell-free systems for production of a recombinant antibody in which the antibody is directed against a target protein that is natively expressed as an intracellular protein. Also provided herein are modified cell-free systems for use in such methods in which the cell free protein expression system is modified by an agent which binds to the intracellular target protein or to the antibody paratope and thus prevents the antibody from binding to the target protein.

### RELATED APPLICATIONS

This application claims priority to U.S. Provisional Patent Application Serial No. 63/398,143 filed on Aug. 15, 2022, which is hereby incorporated into this application in their entirety.

### INCORPORATION OF SEQUENCE LISTING

A computer readable form of the Sequence Listing is filed with this application by electronic submission and is incorporated into this application by reference in its entirety. The Sequence Listing is contained in the file created on May 2, 2023, having the file name MBRACE004.xml, and is 23,837 bytes in size (as measured in the MS-Windows® operating system).

### FIELD

The present disclosure relates to methods and cell-free systems for production of a recombinant antibody, in which the antibody is directed against a target protein that is generally natively expressed as an intracellular protein.

### BACKGROUND

Engineered antibody molecules and their fragments are being increasingly exploited as scientific and clinical tools for therapy and diagnosis of diseases. The unique ability of antibodies to specifically recognize and bind with high affinity to virtually any type of antigen makes them attractive as a starting point for novel biopharmaceutical products and scientific research. Recently certain stress proteins normally present as intracellular proteins have been identified as unique targets for antibody-based therapies in cancer and other pathological conditions since such proteins generally are not expressed, or are expressed at low levels, on the surface of normal cells. Improved methods are needed for producing such antibodies, including antibodies and their fragments that specifically recognize and bind intracellular targets in mammalian cells.

### SUMMARY OF ILLUSTRATIVE EMBODIMENTS

The foregoing general description of the illustrative implementations and the following detailed description thereof are merely exemplary aspects of the teachings of this disclosure, and are not restrictive.

The present disclosure provides, in selected embodiments, methods and cell-free systems for the manufacture of antibodies that bind to intracellular target proteins. The present disclosure also provides methods of producing cell-free systems for the manufacture of antibodies that bind to intracellular target proteins.

The disclosure provides methods of antibody production using cell-free production systems and agents that interfere with the binding between an antibody to be produced using the system and a protein in the cell-free system that is the target protein for the antibody. The binding of the antibody to the target epitope on the target protein within the cell-free system can be disrupted by 1) binding of an agent to the target epitope on the target protein, thus impeding binding of the antibody paratope to the epitope, 2) binding of an agent to the antibody paratope, thus blocking the binding of the antibody paratope to the target epitope, or 3) a combination thereof.

In some embodiments, the disclosure provides methods of recombinant production of an antibody comprising 1) providing a cell-free protein expression system containing a target protein exhibiting an antibody epitope; 2) modifying the cell-free system by introducing one or more agents that block the antibody epitope on the target protein but do not eliminate the activity of the target protein in the cell-free system; 3) introducing one or more nucleic acids encoding an antibody that binds to the antibody epitope to the cell free system ; and 4) initiating transcription and translation of the antibody in the cell-free system under conditions so that the antibody is produced.

In some embodiments, the disclosure provides methods of recombinant production of an antibody comprising 1) providing a cell-free protein expression system containing a target protein exhibiting an antibody epitope; 2) modifying the cell-free system by introducing one or more agents that block an antibody paratope that binds to the antibody epitope on the target protein; 3) introducing one or more nucleic acids encoding an antibody comprising the antibody paratope to the cell free system ; and 4) initiating transcription and translation of the antibody in the cell-free system under conditions so that the antibody is produced. In preferred embodiments, the methods further provide 5) removing the agent that binds the antibody paratope following production of the antibody.

In some embodiments, the cell-free system comprises a cell lysate. In some embodiments, the cell-free system is a eukaryotic cell lysate.

In certain embodiments, the cell lysate is a wheat germ lysate, an insect cell lysate, a reticulocyte lysate, a keratinocyte lysate. In some embodiments, the cell lysate is from a mammalian cell, e.g., from CHO cells, HeLa cells, HEK293 cells, myeloma cells, hybridoma cells or cultivated lymphoma cells.

Thus, in some embodiments, the disclosure provides methods of recombinant production of a monoclonal antibody comprising 1) providing a mammalian cell-free protein expression system containing a target protein exhibiting an antibody epitope; 2) modifying the mammalian cell-free system by introducing one or more agents that block the antibody epitope on the target protein but do not eliminate the activity of the target protein in the cell-free system; 3) introducing one or more nucleic acids encoding a monoclonal antibody that binds to the antibody epitope to the cell free system; and 4) initiating transcription and translation of the monoclonal antibody in the mammalian cell-free system under conditions so that the antibody is produced.

In some embodiments, the cell-free system comprises a cell lysate. In some embodiments, the cell-free system is a eukaryotic cell lysate.

The disclosure provides also provides methods of recombinant production of a monoclonal antibody comprising 1) providing a mammalian cell-free protein expression system containing a target protein exhibiting an antibody epitope; 2) modifying the mammalian cell-free system by introducing one or more agents that block an antibody paratope that binds to the antibody epitope on the target protein; 3) introducing one or more nucleic acids encoding a monoclonal antibody comprising the antibody paratope to the cell free system; and, 4) initiating transcription and translation of the monoclonal antibody in the mammalian cell-free system under conditions so that the antibody is produced. In preferred embodiments, the methods further provide 5) removing the agent that binds to the antibody paratope following production of the monoclonal antibody.

The agent that binds to the target epitope may be introduced at any point prior to the production of the antibody in the cell-free system. For example, if the cell-free system is a cell lysate, the agent may be introduced to the cell prior to the creation of the cell lysate, or alternatively the agent may be introduced to the cell lysate after the lysate has been prepared but prior to the induction of the transcription and translation of the antibody in the system.

The agent that blocks the antibody epitope may be any agent that allows the target protein to retain sufficient function within the cell-free system. In some embodiments the agent is a peptide. In some embodiments the agent is a small molecule. In some embodiments the agent does not affect the activity of the target protein as compared to the target protein activity in the absence of the agent. In some embodiments, the protein activity is reduced but is sufficient to support efficient antibody production in the cell-free system. In some embodiments, the agent reduces the target protein activity about 10% as compared to the target protein activity in the cell-free system in the absence of the agent. In some embodiments, the agent reduces the target protein activity about 50% as compared to the target protein activity in the cell-free system in the absence of the agent.

The disclosure also provides methods of recombinant production of an antibody comprising: 1) introducing an agent that selectively binds to a target epitope on an intracellular protein to cells to create a modified cell line; 2) creating a cell-free antibody production system from the modified cell line; 3) introducing a nucleic acid template to the cell-free antibody production system, wherein the nucleic acid template encodes an antibody that selectively binds to the target epitope on the intracellular protein; and 4) initiating transcription and translation from the nucleic acid template to produce the antibody in the cell-free antibody production system.

In some embodiments, the target protein is an intracellular protein in a secretory pathway. In some embodiments, the target protein is a stress protein. In some embodiments, the target protein is a signaling protein.

In some embodiments, the target protein is expressed on an organelle of the cell line.

In specific embodiments, the target protein is a protein that is generally expressed on the endoplasmic reticulum. For example, the target protein can be an endoplasmic reticulum chaperone, e.g., calreticulin, a heat shock protein or an isomerase. Specifically, the target protein can be glucose-regulated protein 78 (GRP78), HSP47, is protein disulfide isomerase (PDI), calreticulin or GP94.

In specific embodiments, the target protein is generally expressed on the Golgi apparatus. For example, the target protein can be a Golgi complex protein, e.g., Golgi phosphoprotein 2 (GOLPH2), Golgi phosphoprotein 3 (GOLPH3), GM130, ATPase H+ Transporting V1 Subunit A (ATP6V1A), ATPase H+ Transporting V1 Subunit E1 (ATPP6V1E1), ATPase H+ Transporting V0 Subunit A2 (ATP6VOA2), transmembrane protein 165 (TMEM165), Golgin B1 (GOLGB1), SCY1-like 1-binding protein 1 (SCYL1BP1), Trafficking Protein Particle Complex Subunit 11 (TRAPPC11), Trafficking Protein Particle Complex Subunit 2 (TRAPPC2), or Thyroid Hormone Receptor Interactor 11 (TRIP11).

In some embodiments, the target protein is associated with a membrane within the cell.

In some embodiments, the target protein is an intracellular signaling protein, such as those involved in a signal transduction pathway, e.g., a kinase or a phosphatase.

In some embodiments, the antibody is isolated from the cell-free system after production.

Other features, advantages, and aspects will be described below in more detail.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values or dimensions illustrated in the accompanying graphs and figures are for illustration purposes only and may or may not represent actual or preferred values or dimensions. Where applicable, some or all features may not be illustrated to assist in the description of underlying features.

FIG. 1 depicts SDS-PAGE analysis of purified supernatant samples following expression of anti-GRP78 antibody clones B4, D1 and F6 in CHO cells under reducing and non-reducing conditions.

FIG. 2A and FIG. 2B depict flow cytometry results for binding of antibody clones B4 and F6, respectively, to GRP78 variants from an alanine scan library. For each GRP78 variant, the percent mean fluorescence for WT GRP78 binding is shown plotted relative to the control antibody 1H11-1H7.

FIG. 3 depicts visualization of major residues for antibody binding of anti-GRP78 clones B4 and F6 to GRP78, with exemplary major and other residues involved in binding shown by arrows.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

Provided herein are methods of recombinant production of antibodies or binding fragments thereof in which the target protein of the antibody is also generally found as an intracellular protein in cells. In some embodiments, the target intracellular protein to which the antibody binds is a protein involved in the secretory pathway, such as proteins expressed on or in the endoplasmic reticulum (ER) or Golgi apparatus. In some embodiments, the target protein of the antibody is a stress protein, which are proteins that normally function in the ER or Golgi apparatus or other intracellular organelle but that may become upregulated on the surface of cells under conditions of cell stress. For instance, stress proteins, such as those involved in the unfolded protein response (UFR), are proteins found to be upregulated on the surface of cancer cells but not normally found on the surface of normal cells. Stress proteins exhibit desirable features for use as a target protein for antibody-based cancer therapies since such proteins generally are not expressed or are expressed at low levels on the surface of normal cells, whereas they can become upregulated or overexpressed at the surface of cancer cells. The use of stress proteins as a target protein of an antibody-based therapy ensures that their upregulated surface expression in a variety of cancers, but not in normal tissue and cells, and reduces or minimizes off-target activity and/or toxicity.

Methods to produce antibodies in sufficient quantities for use as a therapeutic are required. Several expression systems are available, both from prokaryotic and eukaryotic origin. The choice of system depends on many factors, including the molecular species being expressed and the precise sequence of the individual antibody.

Particularly desirable for recombinant production of human or humanized antibodies are mammalian expression systems. For instance, mammalian cells are able to carry out desired protein folding and post-translational modifications identical to human systems. By expression of recombinant proteins in mammalian systems, such as those derived from HEK293 cells or CHO cells, it is possible to achieve a glycosylation pattern which is similar but not identical to that obtained from human cells. Mammalian cell-free systems allow for very high product yields and are comparatively robust to metabolic stresses.

Although efforts have been made to produce antibodies to stress proteins, it has not been possible to generate them efficiently in mammalian cells. For example, observations herein demonstrate that antibodies directed against the exemplary stress protein GRP78 are not able to be expressed in high yield from mammalian cells without using methods such as those of the present disclosure. Without wishing to be bound by theory, it is believed that, in certain circumstances, stress proteins are required for cell viability or function of the mammalian cell, such that binding of the intracellular protein by an antibody during the course of recombinant antibody production is detrimental to the cell. For example, in some embodiments, binding of certain intracellular proteins inhibits the ability of the intracellular protein to carry out its normal function in the cell, leading to misfolding of proteins in cells, lack of degradation of misfolded proteins, improper calcium homeostasis and, in some cases, a reduction or loss of cell viability. This loss or decrease of function impacts the ability to produce antibody in high yield.

Studies have demonstrated that stress proteins, such as chaperones, are necessary to function properly in a cell during antibody production. For instance, changes in cellular abundance of secretory pathway proteins have been shown to correlate with the ability to produce recombinant monoclonal antibodies abundantly (see e.g., Lambert and Merten, 1997, Biotechnol. Bioeng., 54:165-180; Downham et al. 1996, Biotechnol. Bioeng., 51:691-696). It is known that secretory proteins are folded and assembled into higher order complexes in the cell's inner endoplasmic reticulum (ER) compartment of the cell shortly after protein synthesis. The ER is composed of specific auxiliary assembly factors along with quality control mechanism (Ellgaard et al., Quality control in the secretory pathway, Science. 1999 Dec. 3;286: 1882-8; Helenius et al., Intracellular functions of N-linked glycans, Science. 2001 Mar. 23;291:2364-9). The folding of proteins and the refolding of misfolded soluble and aggregated proteins is known to be mediated by a network of evolutionarily conserved protein molecules called chaperones (Haiti, F. U., Nature, 381, 571-580, (1996); Horwich, A. L., Brooks Low K., Fenton, W. A., Hirshfield, I. N. & Furtak, K., Cell 74, 909-917 (1993); Ellis, R. J. & Hemmingsen, S. M., TiBS, 14, 339-342, (1989); Bukau, B., Hesterkamp, T. & Luirink, J., Trends Cell Biol, 6, 480-486, (1996); Bukau, B., Deuerling, E., Pfund, C. & Craig, E. A., Cell, 101, 119-122, (2000)). Secretory proteins also undergo various post-translational modifications, including glycosylation, during their passage through the Golgi complex.

Alternatively, even if the binding of an intracellular protein by an antibody does not impact cell viability, it can impact the manufacture of the antibody due to sequestration of the product by the intracellular protein. Briefly, the intracellular protein can act as a “sink” for the binding of the antibody or antigen-binding fragment, preventing secretion and thus limiting the yield of the intended production.

The provided methods relate to cell-free systems for production of antibodies or antigen-binding fragments that target an endogenous intracellular target protein in a cell, such as a target protein in the secretory pathway.

The provided production systems and methods are exemplified for antibodies targeting GRP78 and other intracellular target proteins. The systems and methods described herein are generalizable to production of all or part of an antibody-based biologic that selectively targets proteins that are generally found intracellularly, but which can be found on the cell surface in certain pathologies. It will thus be understood by those of ordinary skill in the art that the present disclosure is exemplary and can be applied to other cell systems and manufacturing methods for production of biologics targeting such other targets.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Methods Of Cell Production For Cell-Free Systems

Provided herein is a method of recombinant production of an antibody in which the antibody binds to a target protein (or a homolog thereof) that is normally expressed natively as an intracellular protein. In the provided cell-free systems and methods, the antibody is produced in a modified system, e.g., a mammalian cell-free system, that is modified to contain an agent that selectively binds to an epitope on the target protein to which the antibody binds.

These methods and systems disclosed herein can be used individually or in conjunction with all or specific aspects of other antibody manufacturing systems. In some preferred embodiments, the modified cell line may be produced using a method disclosed in U.S. Ser. No. 63/337,980 filed on May 3, 2022 and/or U.S. Ser. No. 63/359,541 filed on Jul. 8, 2022, each of which are hereby incorporated into this application in their entirety for all purposes. For instance, in some preferred embodiments, the methods of these applications can comprise: a) expressing a nucleic acid encoding an antibody in a modified cell line (in some preferred embodiments a mammalian cell line), wherein the antibody specifically binds to a target epitope on a target protein that is natively expressed as an intracellular protein in the cell; and wherein the modified cell is engineered to express a variant target protein that comprises a mutation of one or more amino acid residues of the target epitope; and, b) culturing the modified cell line under conditions so that the antibody is produced (and, in some preferred embodiments, isolating the antibody). In some preferred embodiments, the methods of these applications can comprise: a) providing an antibody that binds to an epitope on a target protein that is present intracellularly in a cell line (in some preferred embodiments, a mammalian cell line); b) identifying the epitope of the target protein to which the antibody binds; c) generating a modified cell line by mutating the epitope of the target protein in the cell line to reduce or abrogate the binding of the antibody to the epitope on the target protein; d) introducing an expression vector encoding the antibody to the modified cell line; e) culturing the modified cell line under conditions that allow production of the antibody from the expression vector; and, f) expressing a nucleic acid encoding an antibody in the modified cell line (and, in some preferred embodiments, isolating the antibody). In some preferred embodiments, the modified mammalian cell line of these applications comprises a variant protein that is an intracellular protein in the secretory pathway, wherein the variant protein comprises a mutation of a native intracellular protein that is a target protein of an antibody and wherein the mutation is an amino acid substitution(s) of one or more amino acid residues to change a target epitope of the antibody to a mutant epitope. In some preferred embodiments, the methods comprise recombinant production of an antibody targeting GRP78 by: a) expressing a nucleic acid encoding an antibody in a modified mammalian cell line, wherein the antibody binds to GRP78 and wherein the modified mammalian cell is engineered with a variant GRP78 that has a mutant epitope comprising a mutation of one or more amino acid residues of the target epitope of the antibody or antigen binding fragment; and, b) culturing the modified mammalian cell line under conditions so that the anti-GRP78 antibody is produced in the culture supernatant.

### A. Intracellular Protein Targets and Antibodies to Same

In some embodiments, the provided systems and methods are useful for expressing antibodies in which the target protein of the antibody is natively expressed as an intracellular protein in the cell. In particular embodiments, the target protein is a protein that is aberrantly expressed on the surface of cells in cancer and other diseases but that also is necessary for the function of normal cells due to the role of the target protein in normal intracellular biological processes. In some embodiments, the target protein is a protein that is involved in the secretory pathway of cells. In some embodiments, the target protein is expressed on an organelle of the cell, such as in a mammalian cell line. In some embodiments, the target protein is expressed on or in the endoplasmic reticulum. In some embodiments, the target protein is expressed on or in the Golgi apparatus.

In some embodiments, the intracellular protein target is a stress protein. Stress proteins include proteins that are involved in organelle autoregulation to maintain homeostasis to regulate the capacity of the organelle under stress conditions, such as may occur in a tumor environment. Various stress proteins are known and include proteins in the secretory pathway. Various stress proteins, including those resulting from ER stress or Golgi stress, are known: Sasaki and Yoshida, J Biochem. 157:185, 2015; Sasaki and Yoshida, FEBS Letters, 593:2330-2340, 2019; Gao et al., Biofactors, 47:964-974, 2021; Li et al., Mol Neurobiol, 49:1449-59, 2014; Yadav et al., J Cancer Prev., 19:75-88, 2014; and Chen and Cubillos-Ruiz, Nature Reviews Cancer, 21:71-88, 2021.

For example, targets include endoplasmic reticulum chaperones (e.g., calreticulin, heat shock proteins, and isomerases) that can translocate to the cytosol and eventually the surface of cells, particularly during under stress conditions. These proteins have been found to be overexpressed in certain pathological conditions. See, e.g., Weirsma V R et al., Front. Oncol., Vol 5: Art. 7 (2015); Garg AD, Cancer Immunol Immunother 61:215-21 (2012). The disclosure of the present embodiments and application of the teachings therein allow production of biologics targeting endoplasmic reticulum chaperones, as well as other identified targets, without overly compromising cell viability. Exemplary biologics can include, for example, those targeting HSP47 for treatment of cervical cancer, stomach cancers or autoimmune disease (Yokota S et al., Biochem Biophys Res Commun 303:413-8 (2003); Yamamoto N et al., Int J Oncol 43:1855-63 (2013)); biologics targeting protein disulfide isomerase (PDI) for the treatment of central nervous system cancers, ovarian cancer, brain cancer, prostate cancer and lung cancer (Xu S et al., Free Radic Biol Med 52:993-1002 (2012); Zhang L et al., Cancer Invest 27:45342009); Pan Z et al., Int J Oncol 35:823-8 (2009)); and biologics targeting calreticulin for treatment of various cancers (Zamanian M et al., Pathol Oncol Res 149-54 (2013); Gold L I et al., FASEB J24:665-83 (2010), biologics targeting GP94 (Marzec M. et al., Biochim Biophys Acta. 2012 March; 1823(3): 774-787), and biologics targeting GRP78 (Arap et al. Cancer Cell 6:275-284 (2004); Sato et al. Adv Genet 69:97-114 (2010).

A variety of Golgi complex proteins are known to be involved in various pathologic states, especially cancers and autoimmune disease, and monoclonal antibodies that selectively bind to these organelle proteins are suited for production of monoclonal antibodies using the methods as disclosed herein. Such proteins include, but are not limited to, Golgi phosphoprotein 2 (GOLPH2) (Liu et al., Front Oncol 2021 Dec. 7;11:78386); Golgi phosphoprotein 3 (GOLPH3) (Xing M, et al., Mol Biol Cell 27: 3828-3840, 2016; Scott K L, et al., Nature 459: 1085-1090, 2009); GM130 (Chang S H, et al., Mol Ther 20: 2052-2063, 2012); ATPase H+ Transporting V1 Subunit A (ATP6V1A) (Van Damme T, et al., Am J Hum Genet 100: 216-227, 2017); ATPase H+ Transporting V1 Subunit E1 (ATPP6V1E1) (Id.); ATPase H+ Transporting V0 Subunit A2 (ATP6VOA2) (Kornak U, et al., Nat Genet 40: 32-34, 2008); transmembrane protein 165 (TMEM165) (Rosnoblet C, et al., Hum Mol Genet 22: 2914-2928, 2013); Golgin B1 (GOLGB1) (Katayama K, et al., Biochem Biophys Res Commun 499: 459-465, 2018); SCY1-like 1-binding protein 1 (SCYL1BP1) (Hennies H C, et al, Nat Genet 40: 1410-1412, 2008); Trafficking Protein Particle Complex Subunit 11 (TRAPPC11) (Larson A A, et al, Skelet Muscle 8: 17, 2018); Trafficking Protein Particle Complex Subunit 2 (TRAPPC2) (Davis E E, et al., Clin Genet 85: 359-364, 2014); and, Thyroid Hormone Receptor Interactor 11 (TRIP11) (Smits P, N Engl J Med 362: 206-216, 2010).

In some embodiments, the target protein is GRP78. In some embodiments, the target protein is heat shock protein 47 (HSP47). In some embodiments, the target protein is protein disulfide isomerase (PDI). In some embodiments, the target protein is calreticulin. In some embodiments, the target protein is GP94. In some embodiments, the target protein is GOLPH2. In some embodiments, the target protein is GOLPH3. In some embodiments, the target protein is GM130. In some embodiments, the target protein is ATP6V1A. In some embodiments, the target protein is AATPP6V1E1. In some embodiments, the target protein is ATP6VOA2. In some embodiments, the target protein is transmembrane protein 165 (TMEM165). In some embodiments, the target protein is GOLGB1. In some embodiments, the target protein is SCYL1BP1. In some embodiments, the target protein is TRAPPC11. In some embodiments, the target protein is Thyroid Hormone Receptor Interactor 11 (TRIP11).

A skilled artisan is familiar with antibodies to intracellular protein targets, such as stress proteins. For example, antibodies to GRP78 antibodies include: GRP78-specific mouse monoclonal IgG antibody MAb159 (Ojha and Amaravadi, Pharmacol. Res., 120:258-266, 2017), PAT-SM6 (Ojha and Amaravadi, 2017); anti-GRP78 antibodies described in PCT Publ. No. WO2018/057703, PCT Publ. No. WO2014/153056; PCT Publ. No. WO2008/105560; U.S. Publ. No. US2010/0041074; U.S. Pat. No. 10,259,884; and U.S. Pat. No. 10/851,161. Antibodies to protein disulfide isomerase (PDI) include, but are not limited to, Invitrogen PDI Monoclonal Antibody Clone 12 (Thermo Fisher Scientific Catalog #MA5-43389) or Invitrogen PDI Monoclonal Antibody clone 2F6G12H2 (Thermo Fisher Scientific Catalog #MA5-43389). Antibodies to calreticulin include, for example, mAb FMC 75 (Enzo Life Sciences, Cat. No. ADI-SPA-601) and mAb 16 (BD Transduction laboratories, Cat. No. 612137). Antibodies to GOLPH3 include, for example, Thermo Fisher Monoclonal Antibody Clone 905CT9.1.1 (Thermo Fisher Scientific Catalog #MA5-37626). Each of these antibodies can serve as a template for a humanized antibody, using methods such as those disclosed herein in more detail.

In some embodiments, the antibody is a human antibody. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

In addition to using a non-human monoclonal antibody for humanization purposes, a non-human antibody can be used as a template for the selection of a fully human or near fully-human antibody. One particular method for selecting such human antibodies is the use of phage display technology. This methodology may use antibody screening techniques as described, e.g., in Dower et al., WO 91/17271 and McCafferty et al., WO 92/01047, U.S. Pat. Nos. 5,877,218, 5,871,907, 5,858,657, 5,837,242, 5,733,743 and 5,565,332. In these methods, libraries of phage are produced in which different antibodies are provided on the outer surfaces of the phage. The antibodies are usually displayed on the phage as Fv or Fab fragments. Antibodies with a desired specificity are selected by affinity to a selected epitope of the organelle protein.

In a particular exemplary method, human antibodies that selectively bind to an epitope of an organelle protein can be produced using the technique of Winter, WO 92/20791. In this method, either the heavy or light chain variable region of a non-human monoclonal antibody is used. If a light chain variable region is selected as the starting material, a phage library is constructed in which members display the light chain variable region of a non-human monoclonal antibody and a different heavy chain variable region. The heavy chain variable regions are obtained from a library of rearranged human heavy chain variable regions. A phage showing strong specific binding for an epitope of an organelle protein is selected. The human heavy chain variable region from this phage provides the basis for constructing an optimized phage library in which each phage displays the same heavy chain variable region identified from the first display library and a different light chain variable region. The light chain variable regions are obtained from a library of rearranged human variable light chain regions. Phage that display the variable regions of completely human antibodies that show strong specific binding for the epitope on the organelle protein are selected.

Among the provided antibodies are monoclonal antibodies, including monoclonal antibody fragments.

### B. Identification of the Binding Epitope on the Target Protein

In some embodiments, residues in a target protein important for antibody binding can be mapped to define or identify the epitope or binding domain of the antibody to design and/or develop agents that block the epitope in the methods and systems of the disclosure. In some embodiments, cDNA encoding the target protein is randomly mutated by methods such as by treatment with chemical mutagen, irradiation during replication, passage through error-inducing (mutator) cell lines or by oligonucleotide directed random mutagenesis. In some embodiments, oligonucleotide-directed random mutagenesis is used in which a preselected region of the protein is targeted for random mutagenesis. In some embodiments, the preselected region for random mutagenesis is one which is believed to contain a binding site or epitope. A preliminary assessment of a preselected region can be accomplished by any methods known in the art including hydropathicity analysis of the protein sequence or by crystal structure analysis.

In some embodiments, a peptide-based approach can be used to map an epitope of an antibody. In one approach, peptide display technologies, presenting a library of protein fragments on a microarray or the surface of E. coli or phage and finding out which ones the antibody binds to through microarray scanning or flow cytometry. In another approach, a series of overlapping peptides of the target protein or a preselected region thereof are screened for antibody binding, such as by ELISA or other technique to monitor binding interactions. These techniques have the added benefit that they can also identify peptides that can potentially be used as an agent for binding in the methods and systems of the of the invention, or as the basis for development of the agent.

In some embodiments, the epitope can be predicted computationally. In some embodiments, the epitope is predicted using the PEASE tool (Sela-Culang, J. et al. (2014) Structure 22(4):646-57; Sela-Culang et al. (2015) Bioinformatics 31(8):1313-5). In some embodiments, the predicted epitope is validated experimentally. Methods of experimentally testing antibody epitopes are known in the art, including, for example, validation is performed using an antibody cross blocking assay, by performing an assay comprising screening a library, by performing mutation analysis, by deuterium exchange analysis, by peptide binding assays, and/or by x-ray crystallographic studies. In some embodiments, the validation uses libraries and/or peptides derived from the target protein to assess the importance of specific amino acids in specific positions for binding. In some embodiments, the library comprises a library of mutations to a subset or all of the amino acid residues of the target protein. In some embodiments, the library is a yeast display library. In some embodiments, the library is a phage display library.

In some embodiments, to identify the residues in the epitope on the target intracellular protein to be blocked during production of the antibodies, it is desirable to start with a solved crystal structure of the intracellular protein so that the key contact residue(s) between the antibody and the target protein can be identified. This solved structure can be the solved crystal structure of the intracellular protein alone, or that of the intracellular protein bound to the antibody or the antibody fragment.

However, in many cases, a good molecular model could provide the necessary information. In cases where the molecular model is not sufficient, e.g., if the appropriate structural templates do not exist in structural databases, amino acid swapping experiments can provide information on which residues can be targeted for mutation. Alanine scanning mutagenesis (mutating each residue sequentially to Ala) through those regions could identify the key residue(s) involved in antigen-binding (Cunningham B C and Wells J A, Proc Natl Acad Sci USA. 1991 Apr. 15; 88(8): 3407-3411). If changing a single residue to Ala reduced but did not destroy binding, that position could be targeted for blocking with an agent.

Comparative model building provides a great range of structural templates, and computer programs available ensures that models are becoming increasingly accurate. For example, the solved crystal structure of various proteins has been shown to be very close to the structure predicted by molecular modelling.

As shown herein in for GRP78, once the structure of the protein target has been elucidated, a person of ordinary skill in the art can identify the residues that can be bound to decrease antibody binding to an epitope of an organelle protein while leaving sufficient function of that organelle protein, and thus an epitope that can be blocked while retaining protein function as taught in more detail herein. Methods for making blocking polypeptides and/or other agents are well known to one of ordinary skill in the art.

In some embodiments, the agents used are introduced to cells and do not significantly affect the viability of the cell line used to produced the cell-free system. In some embodiments, the viability of the modified cell line containing the agent is retained under standard culture and passage conditions compared to the cell line without the introduced agent.

In some embodiments, binding of an agent to the target epitope on a target protein blocks the epitope from binding of an antibody to be produced but does not have a measurable effect on the function of the target protein in a cell free system.

In some embodiments, binding of an agent to the target epitope reduces the target protein activity by only about 5%, about 10%, about 15%, about 20%, about 25%, about 30%, about 35%, about 40%, about 45%, about 50%, about 55%, about 60%, about 65, about 70%, about 75%, about 80%, about 85%, about 90%, or about 95% as compared to the activity of the target protein in the cell-free system in the absence of the agent. For example, the activity of chaperone proteins such as GRP78 can be assessed using method such as those taught in Hristozova N, et al., PLoS One. 2016; 11(8): e0161970 and Mymrikov E et al., J Biol Chem. 2017 Jan. 13; 292(2): 672-684. The activity of intracellular kinases can be assessed using techniques such as those taught in Haubrich* and Swinney Curr Drug Discov Technol. 2016; 13(1): 2-15.

In some embodiments, binding of an agent to the target paratope on the antibody blocks the paratope from binding to the target epitope on the target protein but does not have a measurable effect on the function of the target protein in a cell free system.

In some embodiments, binding of the produced antibody to the target protein following modification of the cell-free system is reduced by about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold as compared to the binding of the produced antibody to the target protein in the cell-free system in the absence of the agent.

In some embodiments, the agent provides a reduction in affinity for the antibody to the target protein. In some embodiments, the affinity of the antibody for the target protein is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65, at least about 70%, at least about 75%, at least about 80%, at least about 85%, at least about 90%, at least about 95%, or at least about 99% relative to the affinity of the antibody for the target protein lacking the one or more mutations. In some embodiments, the affinity of the antibody for the target protein is reduced by at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 7.5 fold, at least about 8 fold, at least about 8.5 fold, at least about 9 fold, at least about 9.5 fold, at least about 10 fold, at least about 100 fold, or at least about 1000 fold relative to the affinity of the antibody for the target protein in the absence of the agent. In some embodiments, the affinity of the antibody for the target protein is eliminated in the presence of the agent (e.g., binding of the antibody to the target protein is undetectable).

Methods for measuring antibody affinity for a polypeptide are known in the art, and may include, without limitation, performing an ELISA assay, performing a radioimmunoassay (RIA), performing Surface Plasmon Resonance (SPR), performing thermopheresis, performing a competition assay, and performing isothermal titration calorimetry.

### Modification of Antibody Paratopes in Cell-free Systems

In one aspect of the disclosure, the cell-free systems provided utilize one or more agents that reversibly bind to a paratope of the antibody to be produced, thus effectively reducing or eliminating the binding of the antibody to its target protein in the cell-free system. The agent can be any agent that can be removed from the antibody following production without compromising the ability for the antibody to be used for its intended purpose following production. In specific aspects, the agent used to bind to the paratope of the antibody in the cell-free system is all or a portion of the antigen used to initially produce or discover the antibody.

One example of such antibodies that can be used with reversible paratope binding agents are monoclonal antibodies that bind to their target epitopes under certain physiological conditions but which release their target proteins under mild changes in these conditions. Certain monoclonal antibodies are pH-dependent and capable of binding an antigen at a neutral pH of 7-7.4 but release the antigen under slightly more acidic conditions. See, e.g., Bonvin et al., mAbs 7:2, 294-302; March/April 2015; Biochim Biophys Acta,. 2014 November;1844(11):1943-1950.

In another example, calcium-dependent antigen binding can be used for dissociation of antigens following antibody production in cell-free systems of the disclosure. See, e.g., Hironiwa et al., MAbs. 2016 January; 8(1): 65-73. Such antibodies can be selected for their ability to bind to the target protein in the presence of a calcium ion and to elute in the absence of a calcium ion. Id. This property can be used to bind the antibody to the agent in the cell-free system of the disclosure, and once production is completed the agent can be removed from the antibody by removal of the calcium ion from the system or isolation of the antibody and elution into conditions free of or with low calcium concentration.

These and other exemplary reversible antibody-antigen pairs will be apparent to those of ordinary skill in the art upon reading the present disclosure.

In specific embodiments, a blocking peptide is used as the agent to bind to the antibody paratope. In such embodiments, an excess of peptide is added to the cell-free system to a concentration of an estimated 100- to 500-fold more peptide relative to the predicted antibody production.

### Modified Mammalian Cell-Free Systems For Anti-Grp78 Antibody Production

In some embodiments, the provided methods and cell-free systems can be used to produce antibodies directed to GRP78, also called anti-GRP78 antibodies. The antibodies may include full-length antibodies or antigen-binding fragments thereof. GRP78, also known as heat shock protein-5 (Bspa5/BiP), is part of an evolutionarily conserved, ER-linked stress response mechanism that provides cellular survival signals during environmental and physiologic duress. As a molecular component of the ER chaperoning network, GRP78 is classically involved with the processing of unfolded proteins, however newer insights also place the protein at the cell surface with the potential to influence signal transduction (Lee, 2014, Nature Rev Cancer 1.4(4); 263-276). Retrospective IHC studies have demonstrated that GRP78 expression positively correlates with poor survival in advanced breast cancer (Lee et al., 2006, Cancer Research 66(16): 7849-7853) and recurrence in prostate cancer patients (Daneshmand et al., 2007, Human Pathology 38(10): 1547-1552). Upregulation of GRP78 promotes survival and chemoresistance in both proliferating and dormant breast cancer cells. Synthetic peptides composed of GRP78-binding motifs coupled to a cell death-inducing peptide to promote apoptosis in cancer cells, demonstrating its in vivo accessibility (Arap et al., 2004, Cancer Cell 6(3): 275-284).

In some embodiments, the antibody comprises an antibody or an antigen-binding fragment thereof. In some embodiments, the antigen-binding fragment is selected from the group consisting of a Fab, a single-chain variable fragment (scFv), a single-domain antibody, and a nanobody. In further embodiments, the antibody is a full-length antibody. In some embodiments, the anti-GRP78 antibody is a humanized antibody. In some embodiments, the anti-GRP78 antibody is a human antibody.

In some embodiments, the antibody is an antibody as described in PCT published App. No. WO2018057703. In some embodiments, the anti-GRP78 is the antibody designated as B4. In some embodiments, the anti-GRP78 antibody is the antibody designated D 1 . In some embodiments, the anti-GRP78 antibody is the antibody designated F6. In any of such embodiments, the antibody is a full-length antibody of any of the foregoing or is an antigen-binding fragment thereof. In some embodiments, the anti-GRP78 antibody is a full-length antibody.

In some embodiments, the anti-GRP78 antibody includes a variable heavy (VH) chain and a variable light (VL) chain. In some embodiments, the VH chain comprises the VH CDR1 set forth in SEQ ID NO: 1, the VH CDR2 set forth in SEQ ID NO: 2, the VH CDR3 set forth in SEQ ID NO: 3; and the VL chain comprises the VL CDR1 set forth in SEQ ID NO: 4, the VL CDR2 set forth in SEQ ID NO: 5, and the VL CDR3 set forth in SEQ ID NO: 6. In some embodiments, the VH chain has a sequence of amino acids that has at least 85%, at least 90% or at least 95% sequence identity to the sequence set forth in SEQ ID NO: 19 and the VL chain has at least 85%, at least 90% or at least 95% sequence identity to the sequence set forth in SEQ ID NO: 20. In some embodiments, the VH chain is set forth in SEQ ID NO: 19 and the VL chain is set forth in SEQ ID NO:20.

In some embodiments, the VH chain comprises the VH CDR1 set forth in SEQ ID NO: 7, the VH CDR2 set forth in SEQ ID NO: 8, the VH CDR3 set forth in SEQ ID NO: 9; and the VL chain comprises the VL CDR1 set forth in SEQ ID NO: 10, the VL CDR2 set forth in SEQ ID NO: 11, and the VL CDR3 set forth in SEQ ID NO: 12. In some embodiments, the VH chain has a sequence of amino acids that has at least 85%, at least 90% or at least 95% sequence identity to the sequence set forth in SEQ ID NO: 21 and the VL chain has at least 85%, at least 90% or at least 95% sequence identity to the sequence set forth in SEQ ID NO: 22. In some embodiments, the VH chain is set forth in SEQ ID NO: 21 and the VL chain is set forth in SEQ ID NO:22.

In some embodiments, the VH chain comprises the VH CDR1 set forth in SEQ ID NO: 13, the VH CDR2 set forth in SEQ ID NO: 14, the VH CDR3 set forth in SEQ ID NO: 15; and the VL chain comprises the VL CDR1 set forth in SEQ ID NO: 16, the VL CDR2 set forth in SEQ ID NO: 17, and the VL CDR3 set forth in SEQ ID NO: 18. In some embodiments, the VH chain has a sequence of amino acids that has at least 85%, at least 90% or at least 95% sequence identity to the sequence set forth in SEQ ID NO: 23 and the VL chain has at least 85%, at least 90% or at least 95% sequence identity to the sequence set forth in SEQ ID NO: 24. In some embodiments, the VH chain is set forth in SEQ ID NO: 23 and the VL chain is set forth in SEQ ID NO:24.

In some of any embodiments, both the agent and the anti-GRP78 antibody bind to the same epitope on human GRP78. In some embodiments the human GRP78 protein comprises the amino acid set forth in SEQ ID NO: 25.

In some embodiments, both the agent and the anti-GPR78 antibody bind to a target epitope of GRP78 that includes one or more amino acid residues K113, R261, H265, K268, K271, K272, R279, E329 or D333, with reference to residue numbering of human GRP78 set forth in SEQ ID NO:25.

In some embodiments, both the agent and the anti-GPR78 antibody bind to a target epitope of GRP78 that includes one or more amino acid residues from R261, H265, H329, K271, K272 and D333, with reference to residue numbering of human GRP78 set forth in SEQ ID NO:25. In some embodiments, the target epitope includes amino acid residues R261, H265, H329, K271, K272 and D333, with reference to residue numbering of human GRP78 set forth in SEQ ID NO:25.

In some embodiments, both the agent and the anti-GPR78 antibody bind to a target epitope of GRP78 that includes one or more amino acid residues R261, R279, K113, K268 and K271, with reference to residue numbering of human GRP78 set forth in SEQ ID NO:25. In some embodiments, the target epitope includes amino acid residues R261, H265, H329, K271, K272 and D333, with reference to residue numbering of human GRP78 set forth in SEQ ID NO:25.

In some embodiments, the agent binds to the same epitope on human GRP78 as an antibody containing any of the above sequences (e.g., peptides that have the ability to cross-compete for binding to human GRP78 with any of the anti-GRP78 antibodies as described). For example, Biacore analysis, ELISA assays or flow cytometry may be used to demonstrate cross-competition with any of the described antibodies. The ability of a peptide to inhibit the binding to human GRP78 as the anti-GRP78 antibody demonstrates that the peptide can compete with the anti-GRP78 antibody for binding to human GRP78 and thus is considered to bind to the same epitope of human GRP78.

In some embodiments, the agent binds to one or a combination of amino acids selected from: (i) K113Q, K113S, K113D or K113T; (ii) R261Q, R261S, R261D, R261T or R261A; (iii) H265Q, H256S, H265D or H256T; (iv) K268Q, K268S, K268D or K268T; (v) K271Q, K271S, K271D or K271S; (vi) K272Q, K272S, K271D or K272S; (vii) R279Q, R279S, R279D, R279T or R279A; (viii) E329Q, E329S, E329D, E329T or E329A; or (ix) D333Q, D333 S or D333T. In some embodiments, the agent binds to one or a combination of amino acids selected from: (i) K113Q; (ii) R261Q or R261A; (iii) H265Q; (iv) K268Q; (v) K271Q; (vi) K272Q; (vii) R279Q or R279A; (viii) E329Q or E329A; or (ix) D333Q.

### Cell-Free Systems for Antibody Production

Numerous cell-free systems can be used as the basis for the cell-free production systems of the present disclosure, including but not limited to those disclosed in U.S. Pat. Nos. 11,261,218; 10,774,354; 10,316,322; 10,308,716; 10,190,145; 9,951,366; 9,753,040; 9,617,533; 9,175,327; 9,040,253; 8,778,631;7,871,794; 7,118,883; 7,041,479; and U.S. Pat. App. Nos. 20060233789 and 20040191858.

The cell-free systems can be based on either prokaryotic or eukaryotic origin. Among the prokaryotic systems, extracts based on E. coli are regularly used and are available commercially for production of a diverse range of proteins, including antibodies. Systems based on Bacillus subtilis (Kelwick R, et al., Metab Eng. 2016;38:370-81), Pseudomonas putida (Wang H. Synth Biol. 2018), Streptomyces (Li J, et al., Biotechnol Bioeng. 2017;114:1343-53), and Vibrio parahaemolyticus (Dondapati S K, et al., Eng Life Sci. 2018;18:140-8) have been optimized well at the laboratory level due to the ease of preparation of cell-free lysates. A wide range of detailed protocols are currently available for the preparation of E. coil-based lysates. Among the eukaryotic systems, extracts based on rabbit reticulocyte lysate (RRL), wheat germ, insect Spodoptera frugiperda 21 (Sf21), Chinese hamster ovary (CHO), and cultured human cells are regularly used. For review see, e.g., Dondapati S K et al., BioDrugs (2020) 34:327-348.

For the production of complex proteins requiring post-translational modification, eukaryotic cell-free systems are often but not always preferred.

In some embodiments, cell-free protein synthesis systems can use crude cell extracts prepared from cells having edits as taught in the present disclosure. These cells are grown to the appropriate confluence and the contents removed by lysis followed by many steps of washing to remove the cell debris and genomic DNA (Jin X and Hong S H. Biochem Eng J. 2018;138:156-64; Gregorio N E et al., Methods Protoc. 2019;2:24). These cell extracts can be used immediately or stored for future use (e.g., frozen at −80° C. and thawed prior to use). Such extracts contain all the principal components necessary for transcription and translation, such as aminoacyl-tRNA synthetase (AAS), ribosomes, and factors necessary for elongation, initiation, and transcription. Protein synthesis can be realized by combining cell extracts with necessary substrates like amino acids, energy substrates, nucleic acid templates, cofactors, salts, and nucleotides. Cell-free protein synthesis is a fast protein production system since it does not require transfection or cell culture and lacks cell viability constraints.

In some embodiments, cell-free protein synthesis systems are created using the transcription and translation-related factors that are derived from or based on the genetic modifications to the antibody epitopes in the native proteins taught herein. In these embodiments, the system contains only known proteins and substrates, such as those taught in Shimizu et al. (2001) Nat. Biotechnology, vol. 19, p. 751 and Shimizu et al. (2005) Methods, vol. 36, p. 299. These known components of the translation machinery are purified and added individually along with the DNA template to produce the protein, resulting in a highly controlled system. The protein factors participating in the initiation, elongation, and termination of the protein synthesis process are identified and can be adapted individually to the system's requirements.

In some embodiments, the protein synthesis systems are microbial lysate systems (prokaryotic or eukaryotic) with mammalian proteins added to allow for proper protein folding processing, e.g., folding and/or post-translational modification. In such cases, recombinant proteins (e.g. mammalian proteins) can be added to the systems, and in the case where the protein is the target of the production antibody it can be modified to abrogate antibody binding. For example, if mammalian GRP78 is added to a yeast cell-free production system, the GRP78 can be modified prior to production of the recombinant protein to modify the epitope for the specific GRP78 antibody using the mutations as taught in Example 3. Similarly, recombinant proteins for other intracellular proteins added to a cell-free system can be modified so that an antibody produced using the system will not bind to the protein (or will have reduced bunding to the protein). Methods of producing recombinant proteins are well known in the art and are taught, for example, in the Protein Expression Handbook from Thermo Fisher Scientific, Waltham, MA.

### Cell-Free Protein Synthesis Reaction Formats

Cell-free synthesis using the systems of the disclosure can be performed in different formats. Successful synthesis of different antibody formats, including single-chain variable fragments (scFvs), Fab fragments, as well as complete IgGs, has already been shown in E. coli (Groff D. MAbs. 2014;6:671-8; Yin Get al., MAbs. 2012;4:217-25); Sf21 (Jin X et al., Biochem Eng J. 2018;138:156-64; Stech M et al., J Biotech-nol. 2012;164:220-31); reticulocyte (Odegrip R et al., Proc Natl Acad Sci USA. 2004;101:2806-10), wheat germ and CHO cell-free systems (Thoring L., et al., Sci Rep. 2017;7:17-12188; Stech M. et al., Sci Rep. 2017;7:17-12364; Martin R W et al., ACS Synth Biol. 2017;6:1370-9). Furthermore, the upscaling of cell-free reactions to the liter-scale (Zawada J F et al., Biotechnol Bioeng. 2011;108:1570-8; Yin G, Garces E D, Yang J, Zhang J, Tran C, Steiner A R, et al. MAbs 2012;4:217-25) as well as downscaling (Norred S E et al., J Vis Exp. 2015;11:52616;) and high-throughput applications (Contreras-Llano L E, and Tan C. Synth Biol. 2018) have been demonstrated.

In some embodiments, the synthesis reaction format used is a batch format. The batch-based format is the most commonly used method both in the prokaryotic and eukaryotic systems. This method is relatively fast and cheap, and synthesis can be performed within 1.5-3 hours depending on the system. E. coli-based systems can provide protein yields ranging from 100 μg/mL to 2-3 mg/ml. Although the yields from batch-based eukaryotic systems are comparatively low, membrane proteins are automatically incorporated into microsomal membranes and the functionality can be addressed immediately after the synthesis (Brodel A K et al., PLoS One. 2013;8:2013).

In other embodiments, to further scale up the protein yields via batch-based eukaryotic systems, a repetitive batch-based synthesis format has been proposed where the microsomes incorporating the MP of interest generated in an initial synthesis reaction can be added to a fresh cell-free synthesis reaction that has been depleted of its microsomes (Thoring L. et al., PLoS One. 2016;11:2016; Zemella A, et al., Sci Rep. 2018;8:18-26936.

In other embodiments, a continuous exchange cell-free synthesis platform (CECFSP). In this format, a semi-permeable dialysis membrane separates the reaction chamber and a feed chamber and thereby a feed chamber provides the fresh reaction components and enriches the reaction chamber. In exchange, the inhibitory components accumulated during the reaction are removed (Quast R B et al., Sci Rep. 2016;6:30399; Gurramkonda C t al., Biotechnol Bioeng. 2018;115:1253-64; Dondapati SK et al., PLoS One. 2019; Thoring L. et al., Sci Rep. 2017;7:17-12188). Typically, the CECFSP format prolongs the reaction time and increases the protein yields. Until now, the CECFSP format has been used to increase the protein yield by multiple fold, and is widely used as cell-free platforms.

In addition, advances in bioorthogonal reaction chemistries have paved the way to expand the possibilities for ADC development. The site-specific introduction of non-canonical amino acids into a genetically engineered sequence can be used to create site-specifically labeled ADCs (Axup J Y et al., Proc Natl Acad Sci USA. 2012;109:16101-6). Currently, several ADCs are approved for therapy. To date, all of these ADCs have been generated by coupling of mAbs to the cytotoxic linker-payload via surface-exposed lysines, or partial disulfide reduction and conjugation to free cysteines, which typically results in a controlled but heterogeneous ADC population with varying numbers and positions of drug molecules attached to the mAb (Strop P et al., Chem Biol. 2013;20:161-7). From synthesis to functional testing, cell-free systems can accelerate antibody construct evaluation by a sequential or simultaneous screening. The introduction of non-canonical amino acids expands the chemical repertoire and thus the possibilities to modify and improve antibody-based therapeutics. Advanced labeling technologies allow for a very fast qualitative analysis of drug-to-antibody ratio (DAR), linker, linker/position, drug, drug/position (research application), and allow full control of the ADC design.

In some preferred embodiments, the cell-free systems of U.S. Ser. No. 63/359,871 filed on Jul. 10, 2022, which is incorporated herein in its entirety for all purposes, can comprise methods of recombinant production of an antibody by: a) expressing a nucleic acid encoding an antibody in a modified cell-free system (in some preferred embodiments a eukaryotic cell-free system), wherein the antibody specifically binds to a target epitope on a protein of the system, and wherein the modified cell-free comprises a mutation of one or more amino acid residues of the target epitope on the protein in the system; and, initiating transcription and translation of the antibody in the cell-free system under conditions so that the antibody is produced. In some preferred embodiments, such a cell-free system can comprise a cell lysate (in some preferred embodiments a eukaryotic cell lysate). In some preferred embodiments, the cell-free systems of U.S. Ser. No. 63/359,871 filed on Jul. 10, 2022 can comprise: a) modifying a cell line (in some preferred embodiments, a mammalian cell line) to abrogate binding of an antibody to an intracellular protein in the cells of the cell line, wherein the modification results in expression of a variant target protein that comprises a mutation of one or more amino acid residues of the target epitope of the antibody; b) creating a cell-free antibody production system from the modified cell line; c) introducing a nucleic acid template to the cell-free antibody production system, wherein the nucleic acid template encodes the antibody to the target protein; and, d) initiating transcription and translation from the nucleic acid template to produce the antibody in the cell-free antibody production system (and, in some preferred embodiments, isolating the antibody).

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

### Definitions

As used herein, the singular forms “a,” “an,” and “the” include plural referents unless the context clearly dictates otherwise. For example, “a” or “an” means “at least one” or “one or more.” It is understood that aspects and variations described herein include “consisting of” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

The term “agent” as used herein refers to any binding agent with the ability to selectively bind to and block an epitope on a target protein. Agents for use with the present disclosure include, but are not limited to, peptides, proteins, antibodies or fragments thereof; small molecules; aptamers; peptidomimetics; and pharmacophores.

The term “antibody” as used herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv.

Unless otherwise stated, the term “antibody” should be understood to explicitly encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The term “blocking peptide” as used herein refers to peptides comprised of part or all of the amino acid sequence corresponding to the antibody epitope (i.e. the antigen recognized by the antibody). Blocking peptides will bind specifically to the target antibody, preventing subsequent antibody binding to target epitope. Incubation of an antibody with sufficient blocking peptide occupies antibody binding sites to the target protein epitope in a sample or system, preventing subsequent target protein binding to the antibody paratope in the sample or system.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8;309(3):657-70, (“Aho” numbering scheme); Martin et al., “Modeling antibody hypervariable loops: a combined algorithm,” PNAS, 1989, 86(23):9268-9272, (“AbM” numbering scheme); and Ye et al., “IgBLAST: an immunoglobulin variable domain sequence analysis tool,” Nucleic Acids Res. 2013 July;41(Web Server issue):W34-40, (“IgBLAST numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. The IgBLAST scheme is based on matching to germline V, D and J genes, and can be determined using National Center for Biotechnology Information (NCBI)'s IgBLAST tool.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273,927-948

Under the Kabat numbering scheme, in some embodiments, the CDR amino acid residues in the heavy chain variable domain (VH) are numbered 26-35 (HCDR1), 50-65 (HCDR2), and 95-105 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-35 (HCDR1). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50- 65 (HCDR2), and 95-105 (HCDR3) in a VH; and amino acid residues 24-34 (LCDR1), 50-56 (LCDR2), and 89-97 (LCDR3) in a VL.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes (see e.g., Section II), although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

Tolerable variations of the CDR sequences will be known to those of skill in the art. For example, in some embodiments the polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any CDR amino acid sequence.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, an “epitope” refers to any polypeptide determinant capable of specifically binding to an antibody. In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. In certain embodiments, an epitope may have specific three-dimensional structural characteristics (e.g., a “conformational” epitope) and/or specific charge characteristics. Epitopes can be formed both from contiguous and/or juxtaposed noncontiguous residues (for example, amino acids) of the target molecule. Epitopes formed from contiguous residues (for example, amino acids) typically are retained on exposure to denaturing solvents whereas epitopes formed by tertiary folding typically are lost on treatment with denaturing solvents. An epitope may include but is not limited to at least 3, at least 5 or 8-10 amino acid residues. In some embodiments, an epitope is less than 20 amino acid residues in length, less than 15 residues or less than 12 residues. Two antibodies may bind the same epitope within an antigen if they exhibit competitive binding for the antigen. An epitope can be identified by various scans as well, for example an alanine or arginine scan can indicate one or more residues that the antigen-binding molecule can interact with.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a small guide RNA), or micro-RNA.

An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antibody is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are produced by enzyme digestion of a naturally-occurring intact antibody. In some embodiments, the antibody fragments are scFvs.

A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

The term “humanized” is used to describe antibodies wherein complementarity determining regions (CDRs) from a mammalian animal, e.g., a mouse, are combined with a human framework region. Often polynucleotides encoding the isolated CDRs will be grafted into polynucleotides encoding a suitable variable region framework (and optionally constant regions) to form polynucleotides encoding complete antibodies (e.g., humanized or fully-human), antibody fragments, and the like. Additionally, “humanized” antibodies may be chimeric, human-like, humanized or fully human, in order to reduce their potential antigenicity, without reducing their affinity for their organelle protein target. Chimeric, human-like and humanized antibodies have generally been described in the art.

Humanized antibodies have variable region framework residues that are substantially from a human therapeutic antibody (termed an acceptor antibody) and complementarity determining regions substantially from a mouse-antibody, (referred to as the donor immunoglobulin). See, Queen et al., Proc. Natl. Acad. Sci. USA 86:10029-10033 (1989), WO 90/07861, U.S. Pat. Nos. 5,693,762, 5,693,761, 5,585,089, 5,530,101, and Winter, U.S. Pat. No. 5,225,539. The constant region(s), if present, are also substantially or entirely from a human immunoglobulin. The human variable domains are usually chosen from human antibodies whose framework sequences exhibit a high degree of sequence identity with the murine variable region domains from which the CDRs were derived. The heavy and light chain variable region framework residues can be derived from the same or different human therapeutic antibody sequences. The human therapeutic antibody sequences can be the sequences of naturally occurring human antibodies or can be consensus sequences of several human antibodies. See Carter et al., WO 92/22653. Certain amino acids from the human variable region framework residues are selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid should usually be substituted by the equivalent framework amino acid from the mouse antibody when it is reasonably expected that the amino acid: i) noncovalently binds antigen directly, ii) is adjacent to a CDR region, iii) otherwise interacts with a CDR region (e.g. is within about 6 A of a CDR region), or iv) participates in the VL-VH interface.

Other candidates for substitution are acceptor human framework amino acids that are unusual for a human antibody at that position. These amino acids can be substituted with amino acids from the equivalent position of the mouse donor antibody or from the equivalent positions of more typical human antibodies. Other candidates for substitution are acceptor human framework amino acids that are unusual for a human antibody at that position. The preferred variable region frameworks of humanized antibodies usually show at least 75%, more preferably 80%, and even more preferably 85% sequence identity to a human variable region framework sequence or consensus of such sequences.

In certain examples, a mouse monoclonal antibody can be used as the basis for producing human therapeutic biologics. In one method, for illustration and not limitation, the heavy chain variable V_{H} region is cloned by RT-PCR using mRNA prepared from hybridoma cells. Consensus primers are employed to V_{H} region leader peptide encompassing the translation initiation codon as the 5′ primer and a g2b constant regions specific 3′ primer. The sequences from multiple, independently-derived clones, can be compared to ensure no changes are introduced during amplification. The sequence of the V_{H} region can also be determined or confirmed by sequencing a V_{H} fragment obtained by 5′ RACE RT-PCR methodology and the 3′ g2b specific primer.

The light chain variable V_{L} region of a mouse monoclonal antibody can be cloned in an analogous manner as the V_{H} region. In one approach, a consensus primer set designed for amplification of murine V_{L} regions is designed to hybridize to the V_{L} region encompassing the translation initiation codon, and a 3′ primer specific for the murine Ck region downstream of the V-J joining region. In a second approach, 5′RACE RT-PCR methodology is employed to clone a V_{L} encoding cDNA. The cloned sequences are then combined with sequences encoding human constant regions.

In one approach, the heavy and light chain variable regions are re-engineered to encode splice donor sequences downstream of the respective VDJ or VJ junctions, and cloned into the mammalian expression vector, such as pCMV-hγ1 for the heavy chain, and pCMV-hκ1 for the light chain. These vectors encode human γ1 and Ck constant regions as exonic fragments downstream of the inserted variable region cassette. Following sequence verification, the heavy chain and light chain expression vectors can be co-transfected into COS cells to produce chimeric antibodies. Conditioned media is collected 48 hours post transfection and assayed by western blot analysis for antibody production or ELISA for antigen binding. The chimeric antibodies are preferably humanized as described above.

The heavy and light chain variable regions of chimeric and/or humanized antibodies can be linked to at least a portion of a human constant region of choice. The choice of constant region may be driven by the desired mechanism of action of the antibody, e.g., whether cellular mediated toxicity is desired. For example, isotopes IgG1 and IgG3 have antibody-dependent complement activity and isotypes IgG2 and IgG4 do not. Light chain constant regions can be lambda or kappa. Antibodies can be expressed as tetramers containing two light and two heavy chains, as separate heavy chains, light chains, as Fab, Fab′ F(ab′)₂, and Fv, or as single chain antibodies in which heavy and light chain variable domains are linked through a linker.

The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

As used herein, the term “specifically binds” to a target protein or epitope is a term that is well understood in the art, and methods to determine such specific binding are also well known in the art. A molecule is said to exhibit “specific binding” if it reacts or associates more frequently, more rapidly, with greater duration and/or with greater affinity with a particular target protein than it does with alternative proteins. It is also understood that “specific binding” does not necessarily require (although it can include) exclusive binding. Generally, but not necessarily, reference to binding means preferential binding. “Specificity” refers to the ability of a binding protein to selectively bind an antigen.

As used herein, a “stress protein” refer to a protein that functions in normal cells and may be present at high levels under stressful conditions, such as hypoxia, nutrient deprivation, pH changes, oxidative stress, or other metabolic dysregulation of a cell, such as often occurs in cancer cells. A stress protein includes proteins whose expression is increased when the capacity of a cellular organelle, such as the endoplasmic reticulum (ER) or Golgi apparatus, becomes insufficient. A stress protein of the ER includes a protein associated with the unfolded protein response (UPR) that acts to reduce ER stress and restore homeostasis, a protein associated with endoplasmic-reticulum-associated protein degradation (ERAD), or a protein associated with ER stress-mediated apoptosis. Exemplary ER stress proteins include calreticulin, heat shock proteins, and isomerases. A stress protein of the Golgi apparatus includes proteins involved in post-translational modifications such as glycosylation or that are involved in vesicular transport. Exemplary Golgi stress proteins include, for example, GOLPH3.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The term “paratope”, also known as an antigen-binding site”, is the part of an antibody which recognizes and binds to an epitope.

As used herein the terms “polypeptide”, “peptide” and “protein” are used interchangeably herein to refer to polymers of amino acids of any length. The polymer may be linear, cyclic, or branched, it may comprise modified amino acids, and it may be interrupted by non amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component. As used herein the term “amino acid” refers to either natural and/or unnatural or synthetic amino acids, including glycine and both the D or L optical isomers, and amino acid analogs.

A “vector” is a nucleic acid molecule, preferably self-replicating, which transfers an inserted nucleic acid molecule into and/or between host cells. An “expression vector” is a polynucleotide sequence which, when introduced into an appropriate host cell, can be transcribed and translated into a polypeptide(s). An “expression system” usually connotes a suitable host cell comprised of an expression vector that can function to yield a desired expression product.

The term “recombinant” as applied to a polynucleotide means that the polynucleotide is the product of various combinations of cloning, restriction and/or ligation steps, and other procedures that result in a construct that is distinct from a polynucleotide found in nature.

As used herein the terms “operably linked” or “operatively linked” are used to refer the DNA sequences which are juxtaposed in a manner such that the components so described are in a relationship permitting them to function in their intended manner. For example, a promoter is operably linked to a coding sequence if it controls the transcription of the sequence; or a ribosome binding site is operably linked to a coding sequence if it is positioned so as to permit translation. DNA for a signal sequence (secretory leader) is operably linked to DNA for a polypeptide if it is expressed as a precursor which participates in the secretion of the polypeptide. Generally, operably linked means contiguous.

In some preferred embodiments, this disclosure provides methods of recombinant production of an antibody comprising: providing a cell-free protein expression system containing a target protein exhibiting an antibody epitope; modifying the cell-free system by introducing one or more agents that block the antibody epitope on the target protein but do not eliminate the activity of the target protein in the cell-free system; introducing one or more nucleic acids encoding an antibody that binds to the antibody epitope to the cell free system; and, initiating transcription and translation of the antibody in the cell-free system under conditions so that the antibody is produced. In some preferred embodiments, the cell-free system comprises a cell lysate. In some preferred embodiments, the agent is introduced to the cells prior to creation of the cell lysate. In some preferred embodiments, the agent is introduced to the cells following the creation of the cell lysate. In some preferred embodiments, the cell lysate is selected from the group consisting of wheat germ lysates, insect cell lysates, reticulocyte lysates, keratinocyte lysates, cell extracts from CHO cells, HeLa cells, myeloma cells, hybridoma cells and cultivated lymphoma cells. In some preferred embodiments, the methods further comprise isolating the antibody from the cell-free system. In some preferred embodiments, the agent that blocks the antibody epitope is a peptide. In some preferred embodiments, the agent that blocks the antibody is a small molecule. In some preferred embodiments, the target protein is an intracellular protein in the secretory pathway. In some preferred embodiments, the target protein is a stress protein. In some preferred embodiments, the target protein is a cell signaling protein. In some preferred embodiments, the agent does not affect the activity of the target protein as compared to the target protein activity in the absence of the agent. In some preferred embodiments, the target protein activity is reduced about 10% as compared to the target protein activity in the cell-free system in the absence of the agent. In some preferred embodiments, the target protein activity is reduced about 50% as compared to the target protein activity in the cell-free system in the absence of the agent.

In some preferred embodiments, this disclosure provides methods of recombinant production of an antibody, the methods comprising: providing a cell-free protein expression system containing a target protein exhibiting an antibody epitope; modifying the cell-free system by introducing one or more agents that block an antibody paratope that binds to the antibody epitope on the target protein; introducing one or more nucleic acids encoding an antibody comprising the antibody paratope to the cell free system; and, initiating transcription and translation of the antibody in the cell-free system under conditions so that the antibody is produced. In some preferred embodiments, the methods further comprise isolating the antibody from the cell-free system. In some preferred embodiments, the methods further comprise removing the agent that binds the antibody paratope following production of the antibody. In some preferred embodiments, the methods further comprise isolating the antibody from the cell-free system. In some preferred embodiments, the antibody is isolated prior to removal of the agent from the antibody. In some preferred embodiments, the cell-free system comprises a cell lysate. In some preferred embodiments, the cell lysate is selected from the group consisting of wheat germ lysates, insect cell lysates, reticulocyte lysates, keratinocyte lysates, cell extracts from CHO cells, HeLa cells, myeloma cells, hybridoma cells and cultivated lymphoma cells. In some preferred embodiments, the agent is introduced to the cell-free system prior to initiating transcription and translation of the antibody. In some preferred embodiments, the agent that blocks the antibody paratope is a peptide. In some preferred embodiments, the target protein is an intracellular protein in the secretory pathway. In some preferred embodiments, the target protein is a stress protein. In some preferred embodiments, the target protein is a cell signaling protein.

In some preferred embodiments, this disclosure provides methods of recombinant production of a monoclonal antibody, the methods comprising: providing a mammalian cell-free protein expression system containing a target protein exhibiting an antibody epitope; modifying the mammalian cell-free system by introducing one or more agents that block the antibody epitope on the target protein but do not eliminate the activity of the target protein in the cell-free system; introducing one or more nucleic acids encoding a monoclonal antibody that binds to the antibody epitope to the cell free system; and, initiating transcription and translation of the monoclonal antibody in the mammalian cell-free system under conditions so that the antibody is produced. In some preferred embodiments, the mammalian cell-free system comprises a cell lysate. In some preferred embodiments, the methods further comprise isolating the antibody from the mammalian cell-free system.

In some preferred embodiments, this disclosure provides methods of recombinant production of a monoclonal antibody comprising: providing a mammalian cell-free protein expression system containing a target protein exhibiting an antibody epitope; modifying the mammalian cell-free system by introducing one or more agents that block an antibody paratope that binds to the antibody epitope on the target protein; introducing one or more nucleic acids encoding a monoclonal antibody comprising the antibody paratope to the cell free system; and, initiating transcription and translation of the monoclonal antibody in the mammalian cell-free system under conditions so that the antibody is produced. In some preferred embodiments, the methods further comprise removing the agent that binds the antibody paratope following production of the monoclonal antibody. In some preferred embodiments, the methods further comprise isolating the monoclonal antibody from the cell-free system. In some preferred embodiments, the monoclonal antibody is isolated prior to removal of the agent from the monoclonal antibody.

In some preferred embodiments, this disclosure provides methods of recombinant production of an antibody, the methods comprising: introducing an agent that selectively binds to a target epitope on an intracellular protein to cells to create a modified cell line; creating a cell-free antibody production system from the modified cell line; introducing a nucleic acid template to the cell-free antibody production system, wherein the nucleic acid template encodes an antibody that selectively binds to the target epitope on the intracellular protein; and, initiating transcription and translation from the nucleic acid template to produce the antibody in the cell-free antibody production system. In some preferred embodiments, the methods further comprise isolating the antibody from the cell-free system. In some preferred embodiments, the cell line is a mammalian cell line. In some preferred embodiments, the target protein that is natively expressed is an intracellular protein in the secretory pathway of the mammalian cell line.

In some preferred embodiments, the modified cell line may be produced using a method disclosed in U.S. Ser. No. 63/337,980 filed on May 3, 2022 and/or U.S. Ser. No. 63/359,541 filed on Jul. 8, 2022, each of which are hereby incorporated into this application in their entirety for all purposes. For instance, in some preferred embodiments, the methods of these applications can comprise: a) expressing a nucleic acid encoding an antibody in a modified cell line (in some preferred embodiments a mammalian cell line), wherein the antibody specifically binds to a target epitope on a target protein that is natively expressed as an intracellular protein in the cell; and wherein the modified cell is engineered to express a variant target protein that comprises a mutation of one or more amino acid residues of the target epitope; and, b) culturing the modified cell line under conditions so that the antibody is produced (and, in some preferred embodiments, isolating the antibody). In some preferred embodiments, the methods of these applications can comprise: a) providing an antibody that binds to an epitope on a target protein that is present intracellularly in a cell line (in some preferred embodiments, a mammalian cell line); b) identifying the epitope of the target protein to which the antibody binds; c) generating a modified cell line by mutating the epitope of the target protein in the cell line to reduce or abrogate the binding of the antibody to the epitope on the target protein; d) introducing an expression vector encoding the antibody to the modified cell line; e) culturing the modified cell line under conditions that allow production of the antibody from the expression vector; and, f) expressing a nucleic acid encoding an antibody in the modified cell line (and, in some preferred embodiments, isolating the antibody). In some preferred embodiments, the modified mammalian cell line of these applications comprises a variant protein that is an intracellular protein in the secretory pathway, wherein the variant protein comprises a mutation of a native intracellular protein that is a target protein of an antibody and wherein the mutation is an amino acid substitution(s) of one or more amino acid residues to change a target epitope of the antibody to a mutant epitope. In some preferred embodiments, the methods comprise recombinant production of an antibody targeting GRP78 by: a) expressing a nucleic acid encoding an antibody in a modified mammalian cell line, wherein the antibody binds to GRP78 and wherein the modified mammalian cell is engineered with a variant GRP78 that has a mutant epitope comprising a mutation of one or more amino acid residues of the target epitope of the antibody or antigen binding fragment; and, b) culturing the modified mammalian cell line under conditions so that the anti-GRP78 antibody is produced in the culture supernatant.

In some preferred embodiments of the methods disclosed herein, the target protein is an intracellular protein in the secretory pathway of the cell line. In some preferred embodiments, the target protein is a stress protein. In some preferred embodiments, the target protein is a cell signaling protein. In some preferred embodiments, the target protein is expressed on an organelle of the cell line. In some preferred embodiments, the organelle is the endoplasmic reticulum or the Golgi apparatus. In some preferred embodiments, the target protein is an endoplasmic reticulum chaperone. In some preferred embodiments, the endoplasmic reticulum chaperone is a calreticulin, a heat shock protein or an isomerase. In some preferred embodiments, the target protein is glucose-regulated protein 78 (GRP78), HSP47, PDI, calreticulin or GP94. In some preferred embodiments, the target protein is glucose-regulated protein 78 (GRP78). In some preferred embodiments, the target protein is a Golgi complex protein. In some preferred embodiments, the Golgi complex protein is GOLPH2, GOLPH3, GM130, ATP6V1A, ATP6V1E1, ATP6VOA2, TMEM165, GOLGB1, SCYL1BP1, TRAPPC11, TRAPPC2 or TRIP11.

Other embodiments are also contemplated herein as would be understood by those of ordinary skill in the art.

### EXAMPLES

The following examples are included for illustrative purposes only and are not intended to limit the scope of the invention.

### Example 1 Production and Purification of Anti-GPRP78 Antibodies in Mammalian Cells

Fully human anti-GRP78 monoclonal antibodies (mAbs) formatted as full-length IgG1 antibodies (Table E1) were individually transfected into Chinese Hamster Ovary (CHO) cells by electroporation in 6-well plates, topped up with fresh media the following day and incubated for 7 days post-transfection. Transfection was carried out using the Neon™ transfection system and Neon™ transfection system reagents (Thermo Fisher Scientific, Waltham, MA) using pre-set settings for CHO cells. Day 7 post-transfection supernatant was loaded onto 1 mL MabSelect PrisimA™ columns to purify the antibody. Antibody concentration and IgG titer in the supernatant was determined using the Octet QK 384 using Protein A biosensors (Molecular Devices, Wokingham, Berkshire, UK), using an IgG1 antibody as standard.

During the culture and at the time of harvest the transfected CHO cells exhibited reduced viability. The supernatant titers for all anti-GRP78 antibodies produced from the CHO cells were low and less than 5 μg/mL (Table E2). To confirm the reduced titer was not specific to CHO cells, the transfections were repeated in Human embryonic kidney 293 (HEK293) cells and antibody was purified from Day 5 supernatant using similar methods, but the titer also remained low (Table E2).

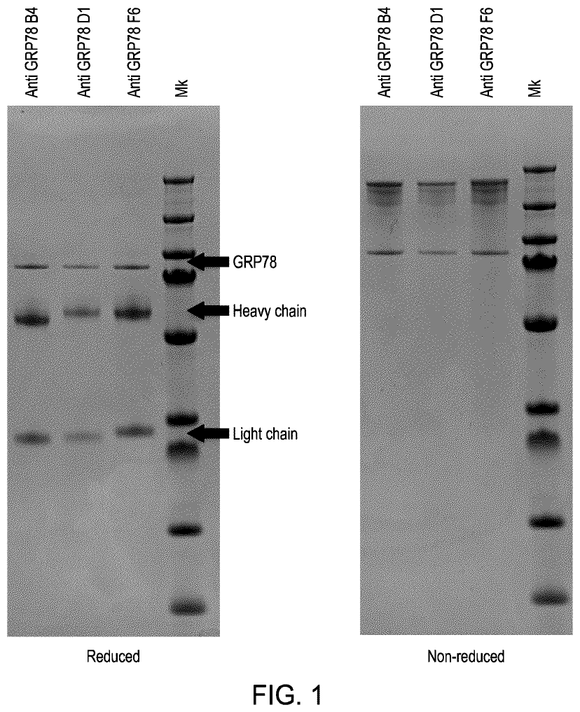

These results are consistent with an observation that production of anti-GRP78 antibody in mammalian cells, such as human cells, is detrimental to the cells and impacts the ability to produce antibody in high yield. It is hypothesized that the antibodies may be binding native GRP78 intracellularly in the endoplasmic reticulum (ER), thereby inhibiting its natural chaperone role in the cell and killing the cells before the antibodies can be secreted into the supernatant for purification. The binding of GRP78 antibodies to the native, ER protein has been demonstrated by experiments showing co-immunoprecipitation of the antibodies with GRP78 as demonstrated by SDS-PAGE analysis of purified sample run under reducing and non-reducing conditions. As shown in FIG. 1, all samples displayed typical SDS-PAGE profile for monomeric IgG, however, there was an extra band between 70-80 kDa consistent with the presence of bound GRP78 antigen.

TABLE E1
Anti-GRP78 Antibodies (disclosed in U.S. Pat. No. 11,254,751)

| | Heavy chain sequences | | | | Light chain sequences | | | |
|---|---|---|---|---|---|---|---|---|
| | (SEQ ID NO) | | | | (SEQ ID NO) | | | |
| | CDR1 | CDR2 | CDR3 | VH | CDR1 | CDR2 | CDR3 | VL |
| B4 | 1 | 2 | 3 | 19 | 4 | 5 | 6 | 20 |
| D1 | 7 | 8 | 9 | 21 | 10 | 11 | 12 | 22 |
| F6 | 13 | 14 | 15 | 23 | 16 | 17 | 18 | 24 |

TABLE E2
Antibody Production in Mammalian Cells

| | | | CHO | HEK |
|---|---|---|---|---|
| | | Total | Day 7 | Day 5 |
| | Concentration | Amount | Titers | Titers |
| Clone | (mg/mL) | (mg) | (μg/mL) | (μg/mL) |
| B4 | 0.799 | 0.80 | 4.0 | 2.1 |
| D1 | 0.098 | 0.06 | 0.9 | Low |
| F6 | 0.497 | 0.30 | 2.5 | 1.3 |

### Example 2 Epitope Mapping of Anti-GRP78 Antibodies

To characterize the binding properties of the antibodies described in Example 1 for GRP78, epitope mapping analysis was carried out by alanine scanning mutagenesis. An alanine scanning library of GRP78 was constructed. Each anti-GRP78 antibody was then screened for binding to each individual GRP78 variant, allowing identification of the target protein residues involved in antibody binding.

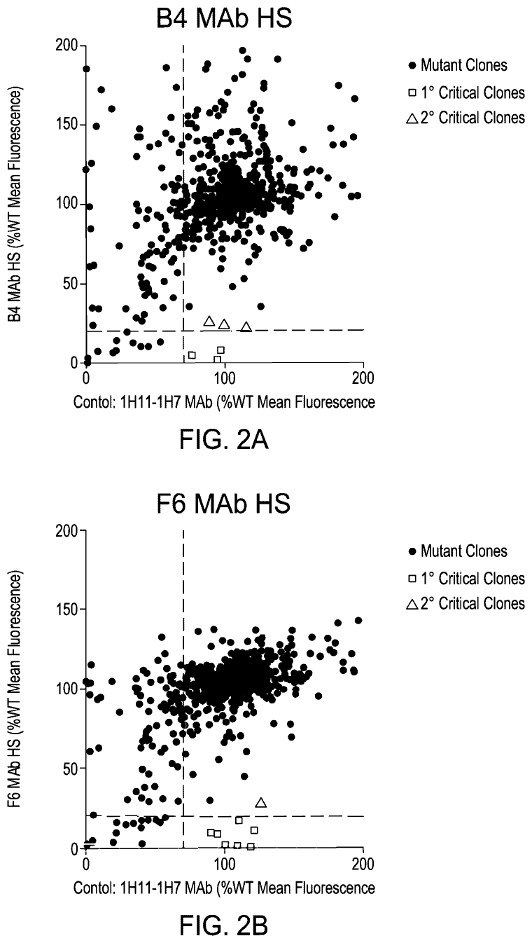

Binding of each test antibody to each GRP78 variant in the alanine scanning library was determined by high-throughput flow cytometry under high stringency conditions (e.g., increased pH, increased salinity, increased temperature and/or increased wash time). Also included in the experiment was a commercially-available monoclonal antibody, 1H11-1H7, which was verified to bind to the wildtype (WT) GRP78 on cells and thus determined to be a suitable positive control under these conditions (Thermo Fisher Scientific, Waltham, MA). Antibody binding to each of the GRP78 variants as measured by the fluorescence signal (raw fluorescence data minus the background), was normalized to its binding to the WT GRP78. For each GRP78 variant, such normalized binding to the test antibodies was plotted relative to that to the control antibody. Exemplary results for two anti-GRP78 antibody clones are shown in FIG. 2A and FIG. 2B. GRP78 variants which exhibited >70% binding to control antibody but <20% binding to the test antibody were identified as primary and major binding residues for the test antibody. Other GRP78 variants which did not meet the criteria above, but showed decreased binding activity (20%-30%) and close proximity (based on the known 3D structure of the protein) to the above identified primary residues, also were identified (and considered as “other residues involved in antibody binding”).

Table E3 sets forth the residues involved in antibody binding for exemplary anti-

GRP78 clones B4 and F6.

TABLE E3
Residues involved in antibody
binding to GRP78

| | |
|---|---|
| B4 | Major: R261, H265, E329 |
| | Others: K271, K272, D333 |
| F6 | Major: R261, R279 |
| | Others: K113, K268, K271 |

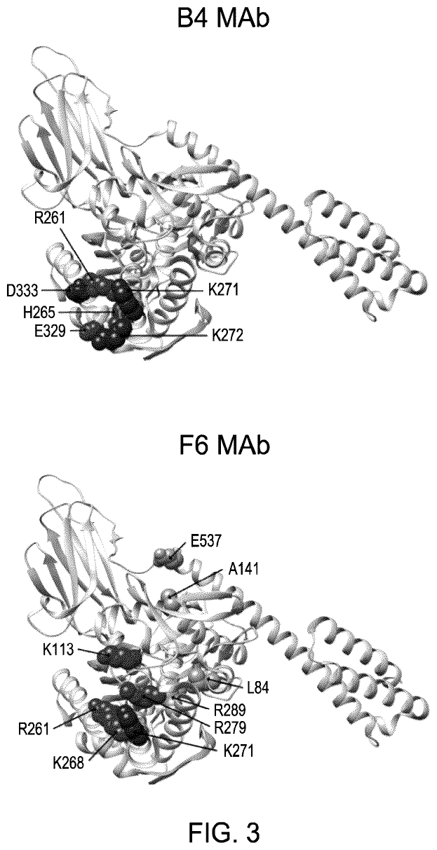

PDB ID EASY (Yang et al., Nature Communications. 2017;8(1):1-3) was examined for identification of the predicted amino acids composing the GRP78 epitopes binding to B4 mAb or F6 mAb. This structure was selected because it is the most complete experimentally-determined structure (spans aa 25-633) of H. sapiens GRP78. (PDB ID #6ASY, Yang et al., 2017). FIG. 3 depicts visualization of major residues for antibody binding, with exemplary major and other residues involved in binding shown by arrows. According to the analyzed structure, all of the amino acids of the predicted epitope are contained in alpha-helices or as part of a linker between two alpha-helices and can be separated by alpha-helix (major residues according to shotgun mutagenesis are underlined): (i) K113; (ii) R261, H265, K268, K271, K272; (iii) R279; and (iv) E329, D333.

### Example 3 Creation of Cell-Free Lysate System from Modified GRP78 Cells

The cell-free systems are created from CHO cells as taught in Stech M, Scientific Reports 7:12030 (2017); Brödel, A. K. et al. PLoS One 8, e82234 (2013) and Thoring, L. et al. PLoS One 11, e0163670 (2016) for the efficient production of anti-GRP78 antibodies. al., Biotechnology and Bioengineering 111, 25-36 (2013).

CHO lysates containing endogenous microsomal vesicles derived from the ER are prepared as described previously (Brödel A. K., et al., (2013) PloS one 8 (12), e82234; Thoring L., et al., (2016). PloS one 11 (9), e0163670). Briefly, the CHO modified as described herein are grown exponentially in bioreactors at 37° C. up to 18×10⁶ cells/ml in chemically defined, serum-free media (PowerCHO™ 2 CD Medium, Lonza, Basel, Switzerland). The cells are collected by centrifugation at 200×g for 15 min, pelleted, washed twice and resuspended in buffer containing 30 mM HEPES-KOH (pH 7.5) and 100 mM NaOAc. Subsequently, the cell suspension is passed through a 20-gauge needle using a syringe which results in the mechanical disruption of the cells. Nuclei and cell debris are removed by a centrifugation step at 6,500×g for 10 min. The obtained supernatant is subjected to a gel filtration step using Sephadex G-25 column (GE Healthcare, Freiburg, Germany) equilibrated in a buffer containing 30 mM HEPES-KOH (pH 7.5) and 100 mM. The filtered supernatant is eluted in lml fractions, and those with an RNA content above an absorbance of 100 at 260 nm are pooled. In order to remove endogenous mRNA, cell lysates are treated with S7 micrococcal nuclease (Roche, Mannheim, Germany) (10 U/ml) and CaCl₂ (1 mM) and are incubated for 20 min at room temperature (RT). Micrococcal S7 nuclease is inactivated by adding EGTA (6.7 mM). Optionally, the CHO lysates are further supplemented with creatine kinase (100 μg/ml). Lysates are shock frozen in liquid nitrogen and subsequently stored at −80° C. until further usage.

GRP78 peptides of approximately 8-12 amino acids containing GRP78 peptide binding motifs that match the binding epitopes of the B4 and H6 binding epitopes as taught in Example 2 are identified using the panning and specificity assays of Blond-Elguindi S Cell 1993 Nov. 19;75(4):717-28 and Arap et al., Cancer Cell. 2004 September;6(3):275-84.

The GRP78 peptides are synthesized and isolated using methods well known in the art. For example, peptides can be ordered from the custom peptide synthesis services at Thermo Fisher Scientific (Waltham, Mass). These peptides are introduced to the CHO cell-free lysate system in an appropriate concentration to provide predicted saturation of the epitope in the system. Activity of GRP78 in the system is assessed using the methods of Hristozova N, et al., PLoS One. 2016; 11(8): e0161970, and the CHO lysates confirmed to have B4 or H6 epitope blockage by treatment of an aliquot of the peptide-treated lysate with the respective antibody. The modified CHO lysate with the best combination of epitope blockage and GRP78 activity is selected for production of the GRP78 antibodies as described below

### Example 4 Production of Antibodies in a GRP78 Modified Cell-Free System

Coupled transcription-translation reactions are then performed as described using the modified CHO lysates of Example 3. Thoring, Id. Translation reactions are composed of 40% (v/v) S7 nuclease-treated CHO lysate containing endogenous microsomal vesicles originating from the ER, HEPES-KOH (pH 7.6, 30 mM, BioMol GmbH, Hamburg, Germany), complete amino acids (100 μM), Mg(OAc)₂ (3.9 mM), KOAc (135 mM, Merck, Darmstadt, Germany), spermidine (0.25 mM, Sigma-Aldrich, St. Louis, United States), energy components (1.75 mM ATP, 0.3 mM GTP, 0.3 mM CTP, 0.3 mM UTP), creatine phosphate (20 mM), T7 polymerase (1 U/μl) (Agilent Technologies, Santa Clara, United States) and ¹⁴C-leucine (to a final concentration of 30 μM); specific radioactivity 46.15 dpm/pmol (PerkinElmer LAS (Germany) GmbH, Rodgau, Germany) in order to allow for the subsequent quantitative and qualitative analysis of cell-free synthesized proteins. Protein synthesis is initiated by addition of DNA template (60 ng/μL). Reactions are incubated for 3 h at 30° C. at 600 rpm in a standard thermomixer (Eppendorf Thermomixer Comfort). Background translational activity is monitored by performing a translation reaction without supplementation of plasmid.

Coding sequences of the anti-GRP78 antibodies are codon-optimized for Cricetulus griseus and equipped with the regulatory sequences necessary to enable in vitro transcription and translation according to Stech et al., (2017). Sci. Rep. 7 (1), 12030. The basic elements are as follows: 5′ untranslated region (UTR): T7 promotor sequence, multiple cloning site (MCS), internal ribosomal entry site (IRES) from the intergenic region (IGR) of the Cricket paralysis virus (CrPV), GCT as start codon; 3′ UTR: T7 terminator sequence, MCS (Id.). DNA templates are synthesized de novo (Agilent, San Jose, CA) and cloned into appropriate vectors (pUC57-1.8k) by Biocat GmbH (Biocat GmbH, Heidelberg). Plasmid preparations for cell-free protein synthesis are prepared using the PureLink®HiPure Plasmid Midiprep Kit (Thermo Fisher Scientific, Waltham, Mass) according to the manufacturer's instructions and subsequently control digested as well as sequenced to verify the correct DNA sequence using an Illumina Miseg™ system (Illumina, San Diego, CA).

Translation reactions are composed of 40% (v/v) S7 nuclease-treated CHO lysate containing endogenous microsomal vesicles originating from the ER, HEPES-KOH (pH 7.6, 30 mM, BioMol GmbH, Hamburg, Germany), complete amino acids (100 μM), Mg(OAc)₂ (3.9 mM), KOAc (135 mM, Merck, Darmstadt, Germany), spermidine (0.25 mM, Sigma-Aldrich, St. Louis, United States), energy components (1.75 mM ATP, 0.3 mM GTP, 0.3 mM CTP, 0.3 mM UTP), creatine phosphate (20 mM), T7 polymerase (1 U/μl) (Agilent Technologies, Santa Clara, United States) and ¹⁴C-leucine (to a final concentration of 30 μM; specific radioactivity 46.15 dpm/pmol (PerkinElmer LAS (Germany) GmbH, Rodgau, Germany) in order to allow for the subsequent quantitative and qualitative analysis of cell-free synthesized proteins. Protein synthesis was initiated by addition of DNA template (60 ng/μL). Reactions were incubated for 3 h at 30° C. at 600 rpm in a standard thermomixer (Eppendorf Thermomixer Comfort). Background translational activity was monitored by performing a translation reaction without supplementation of plasmid.

Following the translation reaction, samples are centrifuged at 16,000×g for 10 min at 4° C. in order to separate the microsomes from the soluble fraction of the translation mixture. The resulting supernatant (first supernatant, or SUP1) is transferred to a fresh reaction tube and stored on ice until further analysis, while the microsomal pellet is resuspended in 1×PBS containing 0.2% n-Dodecyl-β-D-Maltoside (DDM) in order to enable release of translocated and microsome-contained antibodies. Microsomes are resuspended manually by repeated up and down pipetting followed by vortexing and shaking on a vibrax for approximately 45 min. To separate the released proteins from microsomal membrane remnants, a second centrifugation step is performed. The resulting supernatant (second supernatant, or SUP2) is transferred to a fresh reaction tube and stored on ice until further analysis. Reactions supplemented with ¹⁴C-leucine are analyzed by SDS-PAGE followed by autoradiography and liquid scintillation counting, while non-radioactive samples are subjected to functional analysis by enzyme-linked immunosorbent assay (ELISA).

### Example 5 Humanization of an Anti-GOLPH3 Monoclonal Mouse Antibody

GOLPH3 was initially identified as a peripheral membrane protein localized to the trans-Golgi network, but others reported it to be a mitochondrial protein that regulated the mitochondrial mass through the regulation of the mitochondria-specific phospholipid cardiolipin. GOLPH3 has since been implicated in the target of rapamycin (TOR) signaling pathway. GOLPH3-transfected cells enhanced S6 Kinase activity in response to growth factor stimulation by EGF. Simultaneously, AKT phosphorylation increased in these cells, while these events were abrogated in GOLPH3 siRNA treated cells compared to control cells, indicating the GOLPH3 can enhance signaling through TOR-associated complexes. Scott, KL et al., Nature. 2009 Jun. 25; 459(7250): 1085-1090. These results suggest that GOLPH3 is a bona fide oncogene and may be a useful target for therapeutic strategies.

The Thermo Fisher Monoclonal Antibody Clone 905CT9.1.1 (Thermo Fisher Scientific Catalog #MA5-37626, Thermo Fisher Scientific, Waltham, MA), hereafter referred to as “mGOLPH3” is a mouse IgG1 monoclonal antibody that selectively binds to human purified His-tagged GOLPH3 protein. Because GOLPH3 is found primarily in the Golgi and the mitochondria, the methods of the present disclosure are well suited for efficient production of such antibodies in mammalian cells.

The humanized GOLPH3 antibodies for use in the present methods preferably comprise CDR sequences derived from or based on mGOLPH3, as described in more detail herein and in the incorporated references. Briefly, the amino acid sequences of the V_{H} and V_{L} regions of mGOLPH3 are determined using the REmAB® antibody sequencing services provided by Rapid Novor (Ontario, Canada). The CDRs of mGOLPH3 can be determined using the IMGT numbering system as provided, e.g., in Lefranc, M.-P. et al., Dev. Comp. Immunol., 27, 55-77 (2003). Once the CDRs are identified, the mouse monoclonal antibodies can be humanized using methods such as those disclosed in, e.g., Do Couto et al., U.S. Pat. No. 10,613,094, issued Apr. 7, 2020, and Chilcote et al., U.S. Pat. No. 8,673,593, issued Mar. 18, 2014.

### Example 6 Epitope Mapping of the Protein Binding Site of the Anti-GOLPH3 Humanized Antibodies to hGOLPH3

Various methods can be employed for epitope mapping of the binding site of the anti-mGOLPH3 mAb on human hGOLPH3. Exemplary methods are taught in U.S. Pat. No. 11,174,479 to Greenleaf, et al., issued Nov. 16, 2021, and U.S. Pat. No. 11,061,036 to Wilson, et al., issued Jul. 13, 2021.

For example, the binding of mGOLPH3 mAb to biotinylated hGOLPH3 peptides that span hGOLPH3 can be determined using the Streptavidin biosensors. Biotinylated peptides spanning the hGOLPH3 protein are loaded at 5 μg/ml for 700 seconds, 300 seconds for baseline recording and then mGOLPH3 mAb association and dissociation with each of the peptides measured for 600 seconds each at multiple concentrations. Double reference sensors are employed in all testing to measure and subtract any observed background signal from nonspecific binding or system noise.

Analysis is performed with ForteBio Data Analysis Software (v8.2). After background subtraction, a 1:1 local kinetic model was fit to the observed association and dissociation curves. Overall, K_{D}, Kₒₙ, K_{off}, and R2 correlation coefficients are determined. Where possible, a global curve fit was also performed for multiple concentrations of the antibody/analyte.

### Example 7 Identification of hGOLPH3 Antibody Epitope to Identify Pharmacophore Binding Agents

To predict the residues important for identification of a binding agent to the target epitope of the antibodies to hGOLPH3, the crystal structure hGOLPH3 and its orthologs can be used. Wood et al. have reported the x-ray crystal structure of hGOLPH3 to a 2.9-Å resolution (Wood et al., J Cell Biol. 2009 Dec. 28; 187(7): 967-975), as well as conserved regions of activity in the structures of hGOLPH3 and its yeast ortholog, Vps74p (Wood et al., Journal of Cell Biology, 209 187:67-75. Additional structural aspects and residues required for specific functions of GOLPH3 can be found in Bergeron J J M et al., Mol Cell Proteomics. 2017 December; 16(12): 2048-2054 and Schmitz K R, et al., Dev. Cell 2008;14:523-534).

Once the residues of the target epitope are predicted, discovery methods such as those taught in US Pat. No. 8,019,550 are used to identify potential pharmacophore agents for use with the methods and systems of the disclosure. Briefly, the antibody epitope in the protein structure is identified, as well as the relevant binding residues in the epitope site (e.g., the pharmacophore points). Pharmacophore agents are then designed that fit within the predicted epitope, both geometrically and in terms of satisfying enough pharmacophore points. Various techniques may be used to improve the design process, for example using linked structures, (e.g., super position with the target as a reference), comparison of the epitope determined by a simulation model with the shape determined by a mapping process, and/or empirical determination of the epitope using epitope mapping techniques such as those described above.

### Example 8 Generation of Cell-Free Systems for anti-GOLPH3 Humanized Antibody Production

The cell-free systems are then created from CHO cells for the efficient production of anti-GOLPH3 antibodies as taught in Stech M, Scientific Reports 7:12030 (2017); Brödel, A. K. et al. PLoS One 8, e82234 (2013) and Thoring, L. et al. PLoS One 11, e0163670 (2016). CHO lysates are prepared from cultured CHO-K1 cells as described in Brödel, A. K. et al., Biotechnology and Bioengineering 111, 25-36 (2013). The pharmacophores developed as taught in Example 7 are introduced to the CHO lysates in a sufficient concentration to bind to the anti-GOLPH3 antibody epitope while not eliminating the activity of GOLPH3 in the CHO lysate system.

CHO lysates containing endogenous microsomal vesicles derived from the ER are prepared as described previously (Brödel A. K., et al., (2013) PloS one 8 (12), e82234; Thoring L., et al., (2016). PloS one 11 (9), e0163670). Briefly, CHO modified as described herein are grown exponentially in bioreactors at 37° C. up to 18×10⁶ cells/ml in chemically defined, serum-free media (PowerCHO™ 2 CD Medium, Lonza, Basel, Switzerland). The cells are collected by centrifugation at 200×g for 15 min, pelleted, washed twice and resuspended in buffer containing 30 mM HEPES-KOH (pH 7.5) and 100 mM NaOAc. Subsequently, the cell suspension is passed through a 20-gauge needle using a syringe which results in the mechanical disruption of the cells. Nuclei and cell debris are removed by a centrifugation step at 6,500×g for 10 min. The obtained supernatant is subjected to a gel filtration step using Sephadex G-25 column (GE Healthcare, Freiburg, Germany) equilibrated in a buffer containing 30 mM HEPES-KOH (pH 7.5) and 100 mM. The filtered supernatant is eluted in lml fractions, and those with an RNA content above an absorbance of 100 at 260 nm are pooled. In order to remove endogenous mRNA, cell lysates are treated with S7 micrococcal nuclease (Roche, Mannheim, Germany) (10 U/ml) and CaCl₂ (1 mM) and are incubated for 20 min at room temperature (RT). Micrococcal S7 nuclease is inactivated by adding EGTA (6.7 mM). Optionally, the CHO lysates are further supplemented with creatine kinase (100 μg/ml). Lysates are shock frozen in liquid nitrogen and subsequently stored at −80° C. until further usage.

GOLPH3 pharmacopohores as developed in Example 7 are used to modify cell-free systems for production of GOLPH3 monoclonal antibodies. Activity of GOLPH3 in the system is assessed by identifying the ability of GOLPH3 in the system to modulate the phosphorylation status of mTOR substrates as taught in Scott et al, supra., and the CHO lysates confirmed to have GOLPH3 antibody epitope blockage by treatment of an aliquot of the peptide-treated lysate with the respective antibody. The modified CHO lysate with the best combination of epitope blockage and GOLPH3 phosphorylation activity is selected for production of the GOLPH3 antibodies as described below.

### Example 9 Production of Antibodies in a GOLPH3 Modified Cell-Free System

Coupled transcription-translation reactions are then performed as described. Thoring, Id. Translation reactions are composed of 40% (v/v) S7 nuclease-treated CHO lysate containing endogenous microsomal vesicles originating from the ER, HEPES-KOH (pH 7.6, 30 mM, BioMol GmbH, Hamburg, Germany), complete amino acids (100 μM), Mg(OAc)₂ (3.9 mM), KOAc (135 mM, Merck, Darmstadt, Germany), spermidine (0.25 mM, Sigma-Aldrich, St. Louis, United States), energy components (1.75 mM ATP, 0.3 mM GTP, 0.3 mM CTP, 0.3 mM UTP), creatine phosphate (20 mM), T7 polymerase (1 U/μl) (Agilent Technologies, Santa Clara, United States) and ¹⁴C-leucine (to a final concentration of 30 μM; specific radioactivity 46.15 dpm/pmol (PerkinElmer LAS (Germany) GmbH, Rodgau, Germany) in order to allow for the subsequent quantitative and qualitative analysis of cell-free synthesized proteins. Protein synthesis was initiated by addition of DNA template (60 ng/μL). Reactions were incubated for 3 h at 30° C. at 600 rpm in a standard thermomixer (Eppendorf Thermomixer Comfort). Background translational activity was monitored by performing a translation reaction without supplementation of plasmid.

Coding sequences of the anti-GOLPH3 antibodies are codon-optimized for Cricetulus griseus and equipped with the regulatory sequences necessary to enable in vitro transcription and translation according to Stech et al., (2017). Sci. Rep. 7 (1), 12030. The basic elements are as follows: 5′ untranslated region (UTR): T7 promotor sequence, multiple cloning site (MCS), internal ribosomal entry site (IRES) from the intergenic region (IGR) of the Cricket paralysis virus (CrPV), GCT as start codon; 3′ UTR: T7 terminator sequence, MCS (Id.). DNA templates are synthesized de novo (Agilent, San Jose, CA) and cloned into appropriate vectors (pUC57-1.8k) by Biocat GmbH (Biocat GmbH, Heidelberg). Plasmid preparations for cell-free protein synthesis are prepared using the PureLink®HiPure Plasmid Midiprep Kit (Thermo Fisher Scientific, Waltham, Mass) according to the manufacturer's instructions and subsequently control digested as well as sequenced to verify the correct DNA sequence using an Illumina Miseg™ system (Illumina, San Diego, CA).

Coupled transcription-translation reactions are then performed as described. Thoring, Id. Translation reactions are composed of 40% (v/v) S7 nuclease-treated CHO lysate containing endogenous microsomal vesicles originating from the ER, HEPES-KOH (pH 7.6, 30 mM, BioMol GmbH, Hamburg, Germany), complete amino acids (100 μM), Mg(OAc)₂ (3.9 mM), KOAc (135 mM, Merck, Darmstadt, Germany), spermidine (0.25 mM, Sigma-Aldrich, St. Louis, United States), energy components (1.75 mM ATP, 0.3 mM GTP, 0.3 mM CTP, 0.3 mM UTP), creatine phosphate (20 mM), T7 polymerase (1 U/μl) (Agilent Technologies, Santa Clara, United States) and ¹⁴C-leucine (to a final concentration of 30 μM; specific radioactivity 46.15 dpm/pmol (PerkinElmer LAS (Germany) GmbH, Rodgau, Germany) in order to allow for the subsequent quantitative and qualitative analysis of cell-free synthesized proteins. Protein synthesis was initiated by addition of DNA template (60 ng/μL). Reactions were incubated for 3 h at 30° C. at 600 rpm in a standard thermomixer (Eppendorf Thermomixer Comfort). Background translational activity was monitored by performing a translation reaction without supplementation of plasmid.

Following the translation reaction, samples are centrifuged at 16,000×g for 10 min at 4° C. in order to separate the microsomes from the soluble fraction of the translation mixture. The resulting supernatant (first supernatant, or SUP1) is transferred to a fresh reaction tube and stored on ice until further analysis, while the microsomal pellet is resuspended in 1×PBS containing 0.2% n-Dodecyl-β-D-Maltoside (DDM) in order to enable release of translocated and microsome-contained antibodies. Microsomes are resuspended manually by repeated up and down pipetting followed by vortexing and shaking on a vibrax for approximately 45 min. To separate the released proteins from microsomal membrane remnants, a second centrifugation step is performed. The resulting supernatant (second supernatant, or SUP2) is transferred to a fresh reaction tube and stored on ice until further analysis. Reactions supplemented with ¹⁴C-leucine are analyzed by SDS-PAGE followed by autoradiography and liquid scintillation counting, while non-radioactive samples are subjected to functional analysis by enzyme-linked immunosorbent assay (ELISA).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | GGSISSGGYY | B4 VH CDR1 |
| 2 | YIYYSGSTYY NPSLES | B4 VH CDR2 |
| 3 | RYSSIDAFEI | B4 VH CDR3 |
| 4 | GGDDIGSKSV H | B4 VL CDR1 |
| 5 | DDGDRPS | B4 VL CDR2 |
| 6 | QVWDSSSDQY V | B4 VL CDR3 |
| 7 | GDSVSSNSAA | D1 VH CDR1 |
| 8 | RTYYRSRWYN DYAVSVES | D1 VH CDR2 |
| 9 | DPYYYDSSGY YYFDAFGI | D1 VH CDR3 |
| 10 | GGNNIGSKSV H | D1 VL CDR1 |
| 11 | DGSDRPS | D1 VL CDR2 |
| 12 | DDSDRPS | D1 VL CDR3 |
| 13 | GDSVSSNSAA | F6 VH CDR1 |
| 14 | RTYYRSKWYN DYAVSVKS | F6 VH CDR2 |
| 15 | DPYYYDSSGY YYFDAFDI | F6 VH CDR3 |
| 16 | GGDNIGSKSV H | F6 VL CDR1 |
| 17 | DDSDRPS | F6 VL CDR2 |
| 18 | QVWDSTSHHV V | F6 VL CDR3 |
| 19 | QVRLQESGPG LVKPSQTLSL TCTVSGGSIS SGGYYWSWIR | B4 VH |
| | QHPGKGLEWI GYIYYSGSTY YNPSLESRVT ISVDTSKNQF | |
| | SLKLSSVTAA DTAVYYCARY | |
| | SSIDAFEIWG QGTMVTVSS | |
| 20 | SYVLTQPPSV SVAPGKTATI TCGGDDIGSK SVHWYQQKPG | B4 VL |
| | QAPVLVVYDD GDRPSGIPER FSGSNSGNTA TLAISRVEAG | |
| | DEADYYCQVW DSSSDQYVFG | |
| | SGTKLTVL | |
| 21 | QVQLQQSGPG LVEPSQTLSL TCAISGDSVS SNSAAWNWIR | D1 VH |
| | QSPSRGLEWL GRTYYRSRWY NDYAVSVESR ITINPDTSKN | |
| | QFSLQLNSVT PEDTAVYYCA RDPYYYDSSG YYYFDAFGIW | |
| | GQGTMVTVSS | |
| 22 | SYELIQPPSV SVAPGQTARI ACGGNNIGSK SVHWYQQKPG | D1 VL |
| | QAPVLVVYDD SDRPSGIPER FSGSNSGNTA TLTISRVEAG | |
| | DEADYYCQVW DSSSDHPGVF GTGTKLTVL | |
| 23 | QVQLQQSGPG LVKPPQTLSL TCAISGDSVS SNSAAWNWIR | F6 VH |
| | QSPSRGLEWL GRTYYRSKWY NDYAVSVKSR ITINPDTSKN | |
| | QFSLQLNSVT PEDTAVYYCA RDPYYYDSSG YYYFDAFDIW | |
| | GQGTMVTVSS | |
| 24 | SYELTQPHSV SVAPGQTARI TCGGDNIGSK SVHWYQQRPG | F6 VL |
| | QAPVLVVYDD SDRPSGIPER FSGSNSENTA TLTISGVEAG | |
| | DEADYYCQVW DSTSHHVVFG GGTKLTVL | |
| 25 | MKLSLVAAMLLLLSAARAEEEDKKEDVGTVVGIDLGTTYSCV | Wild-Type |
| | GVFKNGRVEIIANDQGNRITPSYVAFTPEGERLIGDAAKNQLTS | Human GRP78 |
| | NPENTVFDAKRLIGRTWNDPSVQQDIKFLPFKVVEKKTKPYIQ | |
| | VDIGGGQTKTFAPEEISAMVLTKMKETAEAYLGKKVTHAVVT | |
| | VPAYFNDAQRQATKDAGTIAGLNVMRIINEPTAAAIAYGLDK | |
| | REGEKNILVFDLGGGTFDVSLLTIDNGVFEVVATNGDTHLGGE | |
| | DFDQRVMEHFIKLYKKKTGKDVRKDNRAVQKLRREVEKAKR | |
| | ALSSQHQARIEIESFYEGEDFSETLTRAKFEELNMDLFRSTMKP | |
| | VQKVLEDSDLKKSDIDEIVLVGGSTRIPKIQQLVKEFFNGKEPS | |
| | RGINPDEAVAYGAAVQAGVLSGDQDTGDLVLLDVCPLTLGIE | |
| | TVGGVMTKLIPRNTVVPTKKSQIFSTASDNQPTVTIKVYEGER | |
| | PLTKDNHLLGTFDLTGIPPAPRGVPQIEVTFEIDVNGILRVTAE | |
| | DKGTGNKNKITITNDQNRLTPEEIERMVNDAEKFAEEDKKLKE | |
| | RIDTRNELESYAYSLKNQIGDKEKLGGKLSSEDKETMEKAVEE | |
| | KIEWLESHQDADIEDFKAKKKELEEIVQPIISKLYGSAGPPPTG | |
| | EEDTAEKDEL | |

What is claimed:

1. A method of recombinant production of an antibody comprising:
a) providing a cell-free protein expression system containing a target protein exhibiting an antibody epitope;
b) modifying the cell-free system by introducing one or more agents that block the antibody epitope on the target protein but do not eliminate the activity of the target protein in the cell-free system;
c) introducing one or more nucleic acids encoding an antibody that binds to the antibody epitope to the cell free system; and,
d) initiating transcription and translation of the antibody in the cell-free system under conditions so that the antibody is produced.

2. The method of claim 1, wherein the cell-free system comprises a cell lysate.

3. The method of claim 2, wherein the agent is introduced to the cells prior to creation of the cell lysate.

4. The method of claim 2, wherein the agent is introduced to the cells following the creation of the cell lysate.

5. The method according to claim 4 wherein the cell lysate is selected from the group consisting of wheat germ lysates, insect cell lysates, reticulocyte lysates, keratinocyte lysates, cell extracts from CHO cells, HeLa cells, myeloma cells, hybridoma cells and cultivated lymphoma cells.

6. The method of claim 1, further comprising e) isolating the antibody from the cell-free system.

7. The method of claim 1, wherein the agent that blocks the antibody epitope is a peptide.

8. The method of claim 1, wherein the agent that blocks the antibody is a small molecule.

9. The method of claim 1, wherein the target protein is an intracellular protein in the secretory pathway.

10. The method of claim 1, wherein the target protein is a stress protein.

11. The method of claim 1, wherein the target protein is a cell signaling protein.

12. The method of claim 1, wherein the agent does not affect the activity of the target protein as compared to the target protein activity in the absence of the agent.

13. The method of claim 1, wherein the target protein activity is reduced about 10% as compared to the target protein activity in the cell-free system in the absence of the agent.

14. The method of claim 1, wherein the target protein activity is reduced about 50% as compared to the target protein activity in the cell-free system in the absence of the agent.

15. A method of recombinant production of an antibody comprising
a) providing a cell-free protein expression system containing a target protein exhibiting an antibody epitope;
b) modifying the cell-free system by introducing one or more agents that block an antibody paratope that binds to the antibody epitope on the target protein;
c) introducing one or more nucleic acids encoding an antibody comprising the antibody paratope to the cell free system; and,
d) initiating transcription and translation of the antibody in the cell-free system under conditions so that the antibody is produced.

16. The method of claim 1, further comprising e) isolating the antibody from the cell-free system.

17. The method of claim 15, further comprising removing the agent that binds the antibody paratope following production of the antibody.

18. The method of claim 15, further comprising isolating the antibody from the cell-free system.

19. The method of claim 17, wherein the antibody is isolated prior to removal of the agent from the antibody.

20. The method of claim 15, wherein the cell-free system comprises a cell lysate.

21. The method according to claim 20 wherein the cell lysate is selected from the group consisting of wheat germ lysates, insect cell lysates, reticulocyte lysates, keratinocyte lysates, cell extracts from CHO cells, HeLa cells, myeloma cells, hybridoma cells and cultivated lymphoma cells.

22. The method of claim 15, wherein the agent is introduced to the cell-free system prior to initiating transcription and translation of the antibody.

23. The method of claim 15, wherein the agent that blocks the antibody paratope is a peptide.

24. The method of claim 1, wherein the target protein is an intracellular protein in the secretory pathway.

25. The method of claim 1, wherein the target protein is a stress protein.

26. The method of claim 1, wherein the target protein is a cell signaling protein.

27. A method of recombinant production of a monoclonal antibody, the method comprising:
a) providing a mammalian cell-free protein expression system containing a target protein exhibiting an antibody epitope;
b) modifying the mammalian cell-free system by introducing one or more agents that block the antibody epitope on the target protein but do not eliminate the activity of the target protein in the cell-free system;
c) introducing one or more nucleic acids encoding a monoclonal antibody that binds to the antibody epitope to the cell free system; and,
d) initiating transcription and translation of the monoclonal antibody in the mammalian cell-free system under conditions so that the antibody is produced

27. The method of claim 26, wherein the mammalian cell-free system comprises a cell lysate.

28. The method of claim 26, further comprising (c) isolating the antibody from the mammalian cell-free system.

29. A method of recombinant production of a monoclonal antibody comprising
a) providing a mammalian cell-free protein expression system containing a target protein exhibiting an antibody epitope;
b) modifying the mammalian cell-free system by introducing one or more agents that block an antibody paratope that binds to the antibody epitope on the target protein;
c) introducing one or more nucleic acids encoding a monoclonal antibody comprising the antibody paratope to the cell free system; and,
d) initiating transcription and translation of the monoclonal antibody in the mammalian cell-free system under conditions so that the antibody is produced.

30. The method of claim 29, further comprising removing the agent that binds the antibody paratope following production of the monoclonal antibody.

31. The method of claim 29, further comprising isolating the monoclonal antibody from the cell-free system.

32. The method of claim 30, wherein the monoclonal antibody is isolated prior to removal of the agent from the monoclonal antibody.

33. A method of recombinant production of an antibody, the method comprising:
a) introducing an agent that selectively binds to a target epitope on an intracellular protein to cells to create a modified cell line;
b) creating a cell-free antibody production system from the modified cell line;
c) introducing a nucleic acid template to the cell-free antibody production system, wherein the nucleic acid template encodes an antibody that selectively binds to the target epitope on the intracellular protein; and,
d) initiating transcription and translation from the nucleic acid template to produce the antibody in the cell-free antibody production system.

34. The method of claim 33, further comprising e) isolating the antibody from the cell-free system.

35. The method of claim 33, wherein the target protein is an intracellular protein in the secretory pathway of the cell line.

36. The method of claim 33, wherein the target protein is a stress protein.

37. The method of claim 33, wherein the target protein is a cell signaling protein.

38. The method of claim 33, wherein the target protein is expressed on an organelle of the cell line.

39. The method of claim 38, wherein the organelle is the endoplasmic reticulum or the Golgi apparatus.

40. The method of claim 39, wherein the target protein is an endoplasmic reticulum chaperone.

41. The method of claim 40, wherein the endoplasmic reticulum chaperone is a calreticulin, a heat shock protein or an isomerase.

42. The method of claim 40, wherein the target protein is glucose-regulated protein 78 (GRP78), HSP47, PDI, calreticulin or GP94.

43. The method of claim 40, wherein the target protein is glucose-regulated protein 78 (GRP78).

44. The method of claim 38, wherein the target protein is a Golgi complex protein.

45. The method of claim 44, wherein the Golgi complex protein is GOLPH2, GOLPH3, GM130, ATP6V1A, ATP6V1E1, ATP6VOA2, TMEM165, GOLGB 1, SCYL1BP1, TRAPPC 11, TRAPPC2 or TRIP11.

46. The method of claim 33, wherein the cell line is a mammalian cell line.

47. The method of claim 46, wherein the target protein that is natively expressed is an intracellular protein in the secretory pathway of the mammalian cell line.


SEQUENCE LISTING (25 sequences)

SEQ ID NO: 1 - protein, 10 aa, Homo sapiens
GGSISSGGYY

SEQ ID NO: 2 - protein, 16 aa, Homo sapiens
YIYYSGSTYYNPSLES

SEQ ID NO: 3 - protein, 10 aa, Homo sapiens
RYSSIDAFEI

SEQ ID NO: 4 - protein, 11 aa, Homo sapiens
GGDDIGSKSVH

SEQ ID NO: 5 - protein, 7 aa, Homo sapiens
DDGDRPS

SEQ ID NO: 6 - protein, 11 aa, Homo sapiens
QVWDSSSDQYV

SEQ ID NO: 7 - protein, 10 aa, Homo sapiens
GDSVSSNSAA

SEQ ID NO: 8 - protein, 18 aa, Homo sapiens
RTYYRSRWYNDYAVSVES

SEQ ID NO: 9 - protein, 18 aa, Homo sapiens
DPYYYDSSGYYYFDAFGI

SEQ ID NO: 10 - protein, 11 aa, Homo sapiens
GGNNIGSKSVH

SEQ ID NO: 11 - protein, 7 aa, Homo sapiens
DGSDRPS

SEQ ID NO: 12 - protein, 7 aa, Homo sapiens
DDSDRPS

SEQ ID NO: 13 - protein, 10 aa, Homo sapiens
GDSVSSNSAA

SEQ ID NO: 14 - protein, 18 aa, Homo sapiens
RTYYRSKWYNDYAVSVKS

SEQ ID NO: 15 - protein, 18 aa, Homo sapiens
DPYYYDSSGYYYFDAFDI

SEQ ID NO: 16 - protein, 11 aa, Homo sapiens
GGDNIGSKSVH

SEQ ID NO: 17 - protein, 7 aa, Homo sapiens
DDSDRPS

SEQ ID NO: 18 - protein, 11 aa, Homo sapiens
QVWDSTSHHVV

SEQ ID NO: 19 - protein, 119 aa, Homo sapiens
QVRLQESGPGLVKPSQTLSLTCTVSGGSISSGGYYWSWIRQHPGKGLEWIGYIYYSGSTYYNPSLESRVTISVDTSKNQFSLKLSSVTAADTAVYYCARYSSIDAFEIWGQGTMVTVSS

SEQ ID NO: 20 - protein, 108 aa, Homo sapiens
SYVLTQPPSVSVAPGKTATITCGGDDIGSKSVHWYQQKPGQAPVLVVYDDGDRPSGIPERFSGSNSGNTATLAISRVEAGDEADYYCQVWDSSSDQYVFGSGTKLTVL

SEQ ID NO: 21 - protein, 130 aa, Homo sapiens
QVQLQQSGPGLVEPSQTLSLTCAISGDSVSSNSAAWNWIRQSPSRGLEWLGRTYYRSRWYNDYAVSVESRITINPDTSKNQFSLQLNSVTPEDTAVYYCARDPYYYDSSGYYYFDAFGIWGQGTMVTVSS

SEQ ID NO: 22 - protein, 109 aa, Homo sapiens
SYELIQPPSVSVAPGQTARIACGGNNIGSKSVHWYQQKPGQAPVLVVYDDSDRPSGIPERFSGSNSGNTATLTISRVEAGDEADYYCQVWDSSSDHPGVFGTGTKLTVL

SEQ ID NO: 23 - protein, 130 aa, Homo sapiens
QVQLQQSGPGLVKPPQTLSLTCAISGDSVSSNSAAWNWIRQSPSRGLEWLGRTYYRSKWYNDYAVSVKSRITINPDTSKNQFSLQLNSVTPEDTAVYYCARDPYYYDSSGYYYFDAFDIWGQGTMVTVSS

SEQ ID NO: 24 - protein, 108 aa, Homo sapiens
SYELTQPHSVSVAPGQTARITCGGDNIGSKSVHWYQQRPGQAPVLVVYDDSDRPSGIPERFSGSNSENTATLTISGVEAGDEADYYCQVWDSTSHHVVFGGGTKLTVL

SEQ ID NO: 25 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL